(19) United States
(12) Patent Application Publication
(10) Pub. No.: US 2026/0243691 A1
(43) Pub. Date: Aug. 20, 2026

(54) Octopus-Inspired Deception and Signaling Systems from an Exceptionally-Stable Acene Variant

(71) Applicant: The Regents of the University of California, Oakland, CA (US)

(72) Inventors: Alon A. Gorodetsky, Irvine, CA (US); Preeta Pratakshya, Oakland, CA (US); Chengyi Xu, Oakland, CA (US); David Joshua Dibble, Santa Ana, CA (US); Panyiming Liu, Oakland, CA (US); Anthony Burke, Fountain Valley, CA (US)

(73) Assignee: The Regents of the University of California, Oakland, CA (US)

(21) Appl. No.: 19/000,597

(22) Filed: Dec. 23, 2024

Related U.S. Application Data
(60) Provisional application No. 63/613,669, filed on Dec. 21, 2023.

(51) Int. Cl. G01N 21/78; C07D 471/06; G01N 21/27

(52) U.S. Cl. CPC G01N 21/78; C07D 471/06; G01N 21/27

(57) ABSTRACT

A family of versatile and exceptionally stable organic molecules—nonacene-like molecule—is described, as well as films and systems/devices comprising the same as an active component, and methods of manufacture and use thereof. The nonacene-like molecule and the systems comprising thereof are characterized by unprecedented optical properties, stability, and ease of manufacturing. In particular, the nonacene-like molecule endows the systems that incorporate it as the active component with adaptive visible and near infrared optical and fluorescent capabilities. An actuator-type deception and signaling device incorporating the nonacene-like molecule and its protonated version, as well as the device's functionality is also described.

### CROSS-REFERENCED APPLICATIONS

This application claims priority to U.S. Provisional Application 63/613,669 filed on Dec. 21, 2023, the disclosure of which is incorporated by reference in its entirety.

### STATEMENT REGARDING FEDERALLY SPONSORED RESEARCH OR DEVELOPMENT

This invention was made with government support under cooperative grants W911NF-16-2-0077 and D16AP00034 awarded by the Defense Advanced Research Projects Agency; FA2386-14-1-3026 awarded by the Air Force Office of Scientific Research; and under grants N00014-17-1-2564 and N00014-21-1-2143 awarded by the Office of Naval Research. The government has certain rights in the invention.

### FIELD OF THE INVENTION

This application generally refers to a family of versatile and exceptionally stable organic molecules characterized by exceptional optical properties, stability, and ease of manufacturing; as well as films, systems, platforms and devices with adaptive visible and near-infrared optical and fluorescent capabilities comprising thereof; and methods of manufacturing and using such platforms in deception and signaling applications.

### BACKGROUND

Multifunctional platforms that can dynamically modulate their color and appearance have attracted widespread attention for applications as varied as displays, signaling, camouflage, anti-counterfeiting, sensing, biomedical imaging, energy conservation, and robotics. Such technologically valuable platforms typically exhibit dynamic spectroscopic properties that emerge from programming of their constituent materials for responsiveness to specific external stimuli (e.g., mechanical, electrical, magnetic, chemical, or thermal inputs). Within this context, the development of systems with tunable spectroscopic and fluorescent properties that span the ultraviolet (UV), visible (Vis), and near-infrared spectral (NIR) regions of the electromagnetic spectrum has remained exceedingly challenging, especially for notoriously demanding tandem deception/camouflage and signaling applications, because of frequently competing materials and device design requirements. Indeed, relatively few classes of camouflage systems with the potential for achieving such desirable capabilities have been explored to date, including dual-band electrochromic devices, metamaterials/metasurfaces electroactive polymers, reconfigurable photonic gels, and biomolecular films. Still, despite much recent progress, these systems have all consistently struggled with multiple critical drawbacks, such as, for example, complex constituent active material preparation methodologies, sophisticated and expensive clean room fabrication schemes, difficult incorporation of more than one tunable spectroscopic capability, a lack of functionality across multiple spectral bands, the requirement for a single type of actuation strategy, slow switching and response times, degradation or inconsistent performance upon repeated actuation, and/or the inability to autonomously self-repair during operation without user intervention. As such, there remains a demand and need for simpler multifunctional technologies and platforms that can overcome the limitations of the technologies for adaptive color and appearance changes reported to date for applications in signaling, display, sensors, camouflage, anti-counterfeiting, biomedical imaging, energy harvesting, and robotics.

### SUMMARY OF THE INVENTION

Various embodiments are directed to a nonacene-like molecule with a molecular architecture including:
a molecular core, wherein the molecular core is an extended, pi-conjugated, all-carbon nonacene core including nine ortho-annulated benzene rings in a linear arrangement;
four peripheral aromatic rings fused onto the molecular core such as to provide stabilizing multiple aromatic sextets;
one or more nitrogen heteroatoms incorporated within one, two, three, or all four of the four peripheral aromatic rings; and
optionally, one or more pendant, alkyl chain-functionalized phenyl rings attached to one, two, three, or all four of the four peripheral aromatic rings.

In various such embodiments, the nonacene-like molecule is a molecule selected from the group consisting of: TBN, PTBN, and any combination thereof.

Various other embodiments are directed to a color- and appearance-changing film characterized by a surface area and a thickness and including:
a first layer including an optically transparent dielectric elastomer, wherein the first layer has an unrelaxed state wherein the first layer is elastically deformed beyond a relaxed state;
a second layer overlaying the first layer and including a film further including a nonacene-like molecule; and
a third layer overlaying the second layer and including an optically transparent, ion-conducting elastomer; wherein:
   the nonacene-like molecule is an acene with a molecular architecture at least including:
      a molecular core, wherein the molecular core is an extended, pi-conjugated, all-carbon acene core including at least five ortho-annulated benzene rings in a linear arrangement;
      a plurality of peripheral aromatic rings fused onto the molecular core such as to provide stabilizing multiple aromatic sextets;
      one or more heteroatoms incorporated within one or more of the plurality of the peripheral aromatic rings; and
      optionally, one or more pendant, alkyl chain-functionalized phenyl rings attached to one or more of the plurality of the peripheral aromatic rings;
wherein, at least when the first layer is in the relaxed state at least the third layer forms a plurality of geometrically reconfigurable nano-to-micro-structures characterized by a structure density;
wherein, when the first layer is in the relaxed state, the surface area is smaller, the thickness is larger, and the structure density is higher than when the first layer is in the unrelaxed state; and
wherein the surface area, the thickness, and the structure density are altered by the elastic deformation of the first layer into and out of the relaxed state.

In various such embodiments, the optically transparent, ion-conducting elastomer is sulfonated pentablock copolymer.

In still various such embodiments, the nonacene-like molecule includes five ortho-annulated benzene in the molecular core and four peripheral aromatic rings.

In still yet various such embodiments, the nonacene-like molecule includes nine ortho-annulated benzene in the molecular core and four peripheral aromatic rings.

In yet still various such embodiments, the one or more heteroatom is an atom selected from the group consisting of: N, B, O, S, P, Si, and any combination thereof.

In yet various such embodiments, the nonacene-like molecule is a molecule selected from the group consisting of: TBN, PTBN, and any combination thereof.

In still yet various such embodiments, the second layer includes more than one variant or derivative of the nonacene-like molecule, each characterized by distinct optical properties and or coloration.

In various such embodiments, the distinct variants or derivatives of the nonacene-like molecule are arranged into a design or pattern over the film.

In yet various such embodiments, the second layer includes TBN and PTBN arranged into a design or pattern over the film.

In still various such embodiments, the design includes a blue annulus including TBN encompassing a brown ring including PTBN.

Still various embodiments are directed to a system capable of electrically induced color- and appearance-changing including:
an active area characterized by a surface area and a thickness and including:
   a first layer including an optically transparent, ion-conducting elastomer;
   a second layer overlaying the first layer including an optically transparent dielectric elastomer, wherein the second layer has an unrelaxed state wherein the second layer is elastically deformed beyond a relaxed state;
   a third layer overlaying the second layer and including a film further including a nonacene-like molecule; and
   a fourth layer overlaying the third layer and including the same or different optically transparent, ion-conducting elastomer as the first layer; wherein:
      the nonacene-like molecule is an acene with a molecular architecture at least including:
      a molecular core, wherein the molecular core is an extended, pi-conjugated, all-carbon acene core including at least five ortho-annulated benzene rings in a linear arrangement;
      a plurality of peripheral aromatic rings fused onto the molecular core such as to provide stabilizing multiple aromatic sextets;
      one or more heteroatoms incorporated within one or more of the plurality of the peripheral aromatic rings; and
      optionally, one or more pendant, alkyl chain-functionalized phenyl rings attached to one or more of the plurality of the peripheral aromatic rings;
wherein, at least when the second layer is in the relaxed state at least the first and fourth layers form a plurality of geometrically reconfigurable nano-to-micro-structures characterized by a structure density;
wherein, when the second layer is in the relaxed state, the surface area is smaller, the thickness is larger, and the structure density is higher than when the second layer is in the unrelaxed state; and
wherein the surface area, the thickness, and the structure density are altered by the elastic deformation of the second layer into and out of the relaxed state.

In various such embodiments, the optically transparent, ion-conducting elastomer is sulfonated pentablock copolymer.

In still various such embodiments, the nonacene-like molecule includes five ortho-annulated benzene in the molecular core and four peripheral aromatic rings.

In still yet various such embodiments, the nonacene-like molecule includes nine ortho-annulated benzene in the molecular core and four peripheral aromatic rings.

In yet still various such embodiments, the one or more heteroatom is an atom selected from the group consisting of: N, B, O, S, P, Si, and any combination thereof.

In various such embodiments, the nonacene-like molecule is a molecule selected from the group consisting of: TBN, PTBN, and any combination thereof.

In still various such embodiments, the third layer includes more than one variant or derivative of the nonacene-like molecule, each characterized by distinct optical properties and or coloration.

In still yet various such embodiments, the distinct variants or derivatives of the nonacene-like molecule are arranged into a design or pattern over the film.

In yet still various such embodiments, the third layer includes TBN and PTBN arranged into a design or pattern over the film.

In yet various such embodiments, the design includes a blue annulus including TBN encompassing a brown ring including PTBN.

Yet still various embodiments are directed to a method for dynamically changing color and appearance of a system including:
providing the system including:
an active area characterized by a surface area and a thickness and including:
a first layer including an optically transparent, ion-conducting elastomer;
a second layer overlaying the first layer including an optically transparent dielectric elastomer, wherein the second layer has an unrelaxed state wherein the second layer is elastically deformed beyond a relaxed state;
a third layer overlaying the second layer and including a film further including a nonacene-like molecule; and
a fourth layer overlaying the third layer and including the same or different optically transparent, ion-conducting elastomer as the first layer; wherein:
   the nonacene-like molecule is an acene with a molecular architecture at least including:
   a molecular core, wherein the molecular core is an extended, pi-conjugated, all-carbon acene core including at least five ortho-annulated benzene rings in a linear arrangement;
   a plurality of peripheral aromatic rings fused onto the molecular core such as to provide stabilizing multiple aromatic sextets;
   one or more heteroatoms incorporated within one or more of the plurality of the peripheral aromatic rings; and
   optionally, one or more pendant, alkyl chain-functionalized phenyl rings attached to one or more of the plurality of the peripheral aromatic rings;
wherein, at least when the second layer is in the relaxed state at least the first and fourth layers form a plura2lity of geometrically reconfigurable nano-to-micro-structures characterized by a structure density;
wherein, when the second layer is in the relaxed state, the surface area is smaller, the thickness is larger, and the structure density is higher than when the second layer is in the unrelaxed state; and
wherein the surface area, the thickness, and the structure density are altered by the elastic deformation of the second layer into and out of the relaxed state;
actuating the system to elastically deform the second layer such as to produce the color and appearance change of the system.

In various such embodiments, actuating includes applying an electrical bias to the first and fourth layers.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the included figures, which are presented as embodiments of the invention and should not be construed as a complete recitation of the scope of the invention, wherein:

FIG. 1 provides pictures of the H. lunulata octopus reversibly changing skin appearance when flashing its blue rings as a warning display (top), and pictures of an individual blue ring reversibly switching between hidden (left) and exposed (right) states on a contrast-enhancing brown dermal background (bottom), according to prior art.

FIG. 2 provides a schematics of the octopus-inspired deception and signaling systems before (left) and after (right) actuation, wherein the device comprises a top transparent proton-conducting polymer electrode, a nonacene variant-based active layer wherein a wrinkled blue annulus surrounds a flatter brown circle, an underlying acrylic dielectric elastomer membrane, and a bottom transparent proton-conducting polymer electrode (top left and top right insets), and wherein, before actuation, the device features contracted microstructured active region with bright visible appearance, strong near-infrared signature, and large fluorescence signals (bottom left), but after actuation, the device features expanded nanostructured active region with light visible appearance, weak near-infrared signature, and small fluorescence signal (bottom right), in accordance with embodiments of the application.

FIG. 3A shows the molecular architecture of the nonacene-like molecule, wherein the extended all carbon nonacene core results in a near-infrared absorbance, the fused peripheral aromatic rings enhance stability, the nitrogen heteroatoms introduce stimuli responsiveness, and the pendant alkyl-functionalized phenyl rings improve solubility, while FIG. 3B schematically shows the synthetic route for the preparation of nonacene-like molecule 4, wherein the two key steps involve a Lewis acid-mediated aza-Diels-Alder reaction and a base-mediated cyclodehydrohalogenation reaction, in accordance with embodiments of the application.

FIG. 4 shows the HOMO (top left) and LUMO (top right) calculated for the unprotonated tetrabenzononacene TBN, 4, as well as the HOMO (bottom left) and LUMO (bottom right) calculated for PTBN, 4+2H⁺, in accordance with embodiments of the application.

FIG. 5A shows he theoretical UV-Vis-NIR absorption spectra calculated for TBN, 4 (blue dashed line) and PTBN, 4+2H⁺ (brown dashed line) with the inset providing the chemical structures of TBN and PTBN; FIG. 5B provides a table comparison of the reported and calculated excited states, dipoles, and oscillator strengths for nonacene, TBN, and PTBN; FIG. 5C sows the experimental UV-Vis-NIR absorption spectra measured for TBN, 4 (blue solid line) and PTBN, 4+2H⁺ (brown solid line), wherein the pictures of the TBN and PTBN solutions in chloroform are inset, and the theoretical and experimental absorption spectra were normalized to unity for clarity; FIG. 5D shows the experimental UV-Vis-NIR absorption spectra measured for TBN in chloroform at different TFA concentrations (top); as well as the experimental UV-Vis-NIR absorption spectra measured for TBN in chloroform without any added acid (blue solid line), PTBN in chloroform that was protonated with TFA (brown solid line), and TBN in chloroform that was protonated with TFA but then neutralized with NaOH (blue dashed line), wherein the absorption spectra were normalized to unity for clarity; and FIG. 5E provides the experimental UV-Vis-NIR absorption spectra measured for TBN without any added acid (blue line) and for PTBN that was protonated with the acidic NEXAR sulfonated pentablock copolymer (brown line) in a 1:4 (v/v) mixture of Hi Sol 15 Aromatic 150 and VM&P Naphtha 66 solvents, wherein the absorption spectra were normalized to unity for clarity, in accordance with embodiments of the application.

FIG. 6 shows a plot of the normalized peak absorbances as a function of time for TBN (blue circles), PTBN (brown squares), and TMS-pentacene (green triangles) in chloroform, in accordance with embodiments of the application.

FIG. 7 shows the experimental UV-Vis-NIR absorption spectra measured for TBN in chloroform shortly after initial preparation of the compound (blue solid line) and after >2 years of storage in the solid state (blue dashed line) (top); and the experimental UV-Vis-NIR absorption spectra measured for TBN in a 1:4 (v/v) mixture of Hi Sol 15 Aromatic 150 and VM&P Naphtha 66 solvents shortly after initial preparation of the compound (blue solid line) and after >2 years of storage in solution (blue dashed line) (bottom), wherein the absorption spectra were normalized to unity for clarity, in accordance with embodiments of the application.

FIG. 8 provides a schematic of the key steps required during the fabrication of tri-layer architectures with TBN-based central layers (top); and a schematic of the key steps required during the fabrication of tri-layer architectures with PTBN-based central layers, in accordance with embodiments of the application.

FIGS. 9A through 9F provide physical and spectroscopic characterization of films prepared with TBN and PTBN in device-relevant configurations, wherein, FIG. 9A provides on top: schematic (left), pictures (middle), and AFM images (right) of unactuated TBN-based tri-layer architectures, and, on bottom: representative UV-Vis-NIR absorptance (dark blue solid line), transmittance (dark blue dotted line), and reflectance (dark blue dashed-dotted line) spectra for unactuated tri-layer TBN-based architectures;

FIG. 9B provides on top: schematic (left), pictures, (middle), and AFM images (right) of actuated TBN-based tri-layer architectures, and on bottom: representative UV-Vis-NIR absorptance (light blue solid line), transmittance (light blue dotted line), and reflectance (light blue dashed-dotted line) spectra for actuated TBN-based tri-layer architectures;

FIG. 9C provides on top: schematic (left), pictures (middle), and AFM images (right) of unactuated PTBN-based tri-layer architectures, and on bottom: representative UV-Vis-NIR absorptance (dark brown solid line), transmittance (dark brown dotted line), and reflectance (dark brown dashed dotted line) spectra for unactuated PTBN-based tri-layer architectures; FIG. 9D provides on top: schematic (left), pictures (middle), and AFM images (right) of actuated PTBN-based tri-layer architectures, and on bottom: representative UV-Vis-NIR absorptance (light brown solid line), transmittance (light brown dotted line) and reflectance (light brown dashed-dotted line) spectra for actuated PTBN-based tri-layer architectures; FIG. 9E provides on top: schematic (left), pictures (middle), and AFM images (right) of unactuated TBN-based tri-layer architectures fabricated from TBN solutions stored for >2 years, and on bottom: representative UV-Vis-NIR absorptance (dark blue solid line), transmittance (dark blue dotted line), and reflectance (dark blue dashed-dotted line) spectra for unactuated tri-layer TBN-based architectures fabricated from TBN solutions stored for >2 years; and FIG. 9F provides on top: schematic (left), pictures (middle), and AFM images (right) of actuated TBN-based tri-layer architectures fabricated from TBN solutions stored for >2 years; and on bottom: representative UV-Vis-NIR absorptance (light blue solid line), transmittance (light blue dotted line), and reflectance (light blue dashed-dotted line) spectra for actuated TBN-based tri-layer architectures fabricated from TBN solutions stored for >2 years, in accordance with embodiments of the application.

FIG. 10 provides a schematic of the key steps required during the fabrication of quad-layer devices for which the active layers consist of TBN-based blue annuli enclosing PTBN/NEXAR-based brown circles, in accordance with embodiments of the application.

FIG. 11A provides a schematic of a visible appearance-changing system undergoing monitoring during electrical actuation under standard indoor lighting in ambient conditions (top), and representative digital camera images of an appearance-changing device before (left) and after (right) electrical actuation, wherein the device was actuated with a bias of ~2.9 kV (bottom), while FIG. 11B shows the same for a bias of 3.2 kV; FIG. 11C provides on top: plot of the areal strain as a function of the applied voltage for the devices' outer annuli (blue line) and inner circles (brown line), and on bottom: plot of the lightness modulation as a function of the applied voltage for the devices' outer annuli (blue line) and inner circles (brown line), wherein the error bars represent standard deviations; and FIG. 11D provides on top: plot of the areal strain as a function of time for a representative device's outer annulus (blue line) and inner circle (brown line) over three consecutive on/off actuation cycle, and on bottom: plot of the lightness modulation as a function of time for a representative device's outer annulus (blue line) and inner circle (brown line) over three consecutive on/off actuation cycles, in accordance with embodiments of the application.

FIG. 12A provides a plot of the lightness modulation as a function of the actuation cycle number for a representative device's outer annulus (blue line) and inner circle (brown line) over 500 sequential actuation cycles; FIG. 12B provides on top: a schematic of a device that (i) initially continuously operates and features uniform coloration, (ii) spontaneously fails with a pause in function and acquires localized discoloration presumably due to a pinhole short, and (iii) resumes operation after autonomous self-repair with the localized discoloration remaining (from left to right), and on bottom: representative digital camera images of an appearance-changing device during the three stages shown in on top, wherein the breakdown region, which becomes and remains discolored, is indicated with red dashed circles; and the device was actuated with a bias of ~2.9 kV; and FIG. 12C provides plot of the lightness modulation as a function of the actuation cycle number for a representative device's outer annulus (blue line) and inner circle (brown line) over 500 sequential actuation cycles, in accordance with embodiments of the application.

FIGS. 13A through 13C shows signature management and signaling functionalities of TBN- and PTBN-based systems, wherein FIG. 13A provides a schematic of a near-infrared signature management system undergoing monitoring during electrical actuation under near-infrared illumination in ambient atmosphere (top), and a representative spectrally filtered digital camera images of a signature management device before (left) and after (right) electrical actuation with a bias of ~3.2 kV, wherein the changes in area and near-infrared contrast for the annulated circle as a result of electrical actuation; FIG. 13B provides a schematic of a fluorescence signaling system undergoing monitoring during electrical actuation under ultraviolet illumination in ambient atmosphere (top), and a representative digital camera images of a signaling device before (left) and after (right) electrical actuation with a bias of ~3.2 kV, wherein the changes in area and fluorescence signal intensity for the annulated circle as a result of electrical actuation (bottom); while FIG. 13C provides the fluorescence spectra measured for TBN (blue line) and PTBN (brown line) in chloroform at an identical excitation wavelength of 365 nm (top), and the fluorescence spectra measured for TBN (blue line) and PTBN (brown line) in chloroform at different excitation wavelengths of 667 nm and 842 nm, respectively (bottom), in accordance with embodiments of the application.

### DETAILED DESCRIPTION OF THE INVENTION

Turning now to the schemes, images, and data, a nonacene-like molecule with advantageous optical and spectroscopic properties, as well as unprecedented stability and ease of synthetic access, is described, as well as films and systems/devices capable of color and appearance change comprising the same as an active component, and methods of manufacture and use thereof. In particular, in many embodiments, the nonacene-like molecule endows the systems that incorporate it as the active component with adaptive visible and near infrared optical and fluorescent capabilities. In many embodiments, the nonacene-like molecule has a molecular architecture comprising a molecular core, wherein the molecular core is an extended, pi-conjugated, all-carbon nonacene core including nine ortho-annulated benzene rings in a linear arrangement; four peripheral aromatic rings fused onto the molecular core such as to provide stabilizing multiple aromatic sextets; one or more nitrogen heteroatoms incorporated within one, two, three, or all four of the four peripheral aromatic rings; and, optionally, one or more pendant, alkyl chain-functionalized phenyl rings attached to one, two, three, or all four of the four peripheral aromatic rings. In many embodiments, the nonacene-like molecule is a molecule selected from the group consisting of: TBN, PTBN, and any combination thereof. In many embodiments, the nonacene-like molecule is incorporated into a color- and appearance-changing film, system, and or device. It will be understood that the embodiments of the invention described herein are not intended to be exhaustive or to limit the invention to precise forms disclosed. Rather, the embodiments selected for description have been chosen to enable one skilled in the art to practice the invention.

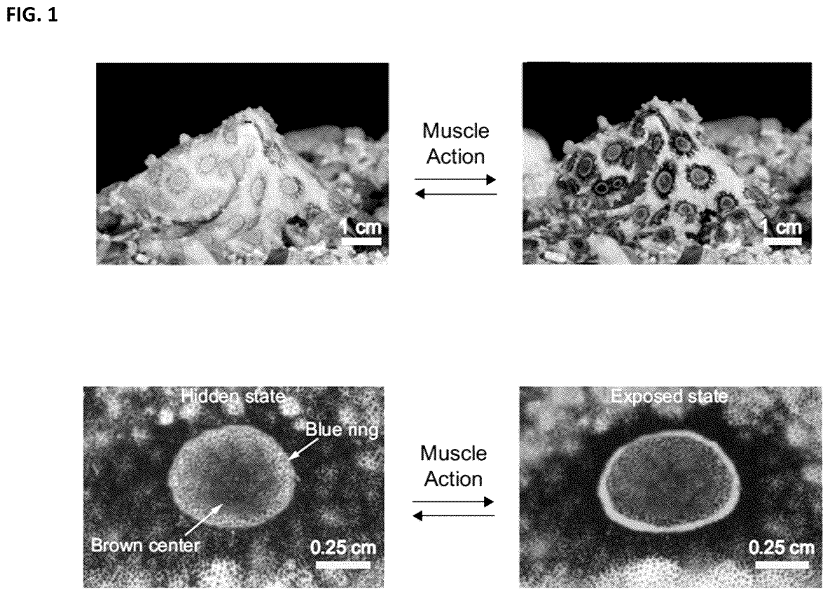

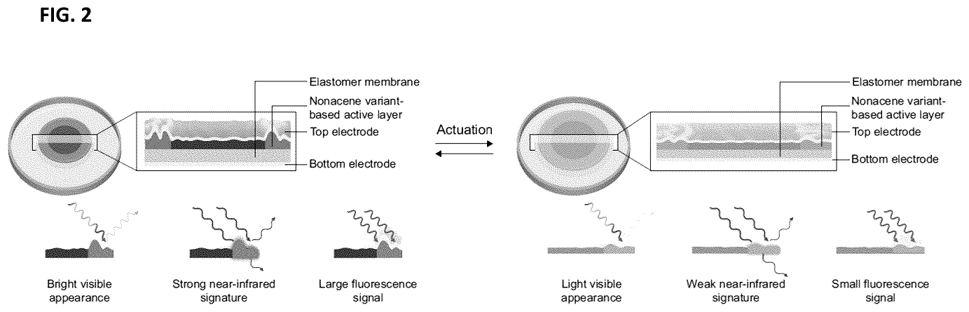

Technologies and platforms that can dynamically and controllably (i.e., adaptively) change color and or appearance are sought after for a wide range of applications including, as some examples, signaling, displays, sensors, camouflage, anti-counterfeiting, sensing, biomedical imaging, energy conservation, and robotics. One notable example of an adaptive color-changing capability found in nature that can serve as an inspiration for adaptive man-made technologies is the dynamic camouflage and aposematic warning display abilities of Hapalochlaena lunulata (H. lunulata) octopus skin (FIG. 1), which are enabled by the muscle-controlled switching of iridescent blue rings between hidden and exposed states on a contrast-enhancing brown background. However, such deception and signaling capability is particularly difficult to reproduce in a laboratory due to a lack of readily available and stable materials with tunable spectroscopic and fluorescent properties that span the UV-Vis-NIR regions of the electromagnetic spectrum.

Nevertheless, within the realm of artificial adaptive deception and signaling platforms, one well-known class of active materials that has received comparatively minimal attention is acenes, which are organic polycyclic aromatic hydrocarbons consisting of linearly fused benzene rings and containing one aromatic sextet. Yet, acenes exhibit a variety of advantageous properties, including straightforward synthetic accessibility, chemically modular pi-conjugated core structures, amenability to peripheral substitution and functionalization, compatibility with bench-top fabrication and processing procedures, theoretically well-understood electronic characteristics, and stimuli-responsive spectroscopic properties. Historically, the shorter acenes and heteroacenes (e.g., pentacene and its variants) have played pivotal roles in (1) organic chemistry as model polycyclic aromatic hydrocarbons and in (2) organic optoelectronics as prototypical active materials for light-emitting diodes, solar cells, and field-effect transistors. More recently, significant research effort has been expended on obtaining and characterizing longer acenes (and, in particular, nonacene derivatives) because of their significance from a fundamental physical organic chemistry viewpoint and their theoretically predicted superior properties for optoelectronic applications (e.g., smaller band gaps, higher mobilities, and tunable visible to near-infrared spectra). However, the solution-based synthesis of classic nonacene or its variants has proven difficult, due to insufficient solid-state stability (of at most two months) even under inert conditions, poor solution-phase stability (of seconds to hours) in ambient atmosphere under light, and limited solubility in many solvent systems. Consequently, the nonacene variants studied to date could neither be processed into thin films, nor used in conventional fabrication schemes, thus limiting their availability for device applications studies and leaving technological potential of these molecules largely unknown. Nevertheless, the recently reported synthesis of nonacene derivatives and nitrogen-containing tetrabenzopentacenes suggests a host of new opportunities for this class of molecules. In addition, separately, the recently reported adaptive infrared camouflage systems also offers a new and promising conceptual approach to dynamically controllable materials and platforms comprising thereof.

This application is directed to embodiments of an organic molecule characterized by exceptional stability, optical properties, and ease of synthesis; as well as films, systems, platforms and devices with adaptive visible and near-infrared optical and fluorescent capabilities comprising thereof and employing thereof as an active material; and methods of manufacturing and using such platforms in deception and signaling applications. In many embodiments, the organic molecule is a designer nonacene-like molecule. In many embodiments, the nonacene-like molecule is a tetrabenzononacene (TBN) or its derivative, such as, for example, protonated TBN (PTBN). In many embodiments, films, systems, platforms and devices are a system comprising the nonacene-like molecule as its active component or material. In many such embodiments, the active component or material of the system is a film comprising the nonacene-like molecule.

In many embodiments, the system is a dielectric elastomer actuator (DEA) device inspired by and, in principle, similar to the natural blue ring system of H. lunulata. In many such embodiments, the device comprises four layers: a first layer comprising a first electrode, wherein the first electrode comprises an ion conductive elastomer; a second layer comprising an active film, wherein the active film further comprises at least one variant or derivative of the nonacene-like molecule; a third layer comprising a dielectric elastomer membrane; and a fourth layer comprising a second electrode, wherein the second electrode comprises the same or different ion conductive elastomer as the first electrode, as schematically illustrated in FIG. 2. In many embodiments, the device is characterized by a nano-to-micro-structured surface texture (i.e., wrinkles) when in the relaxed (unactuated) state, wherein the nano-to-micro-structured texture is flatted out to an extent upon stretching-causing actuation (such as, for example, an electrical bias). In many embodiments, the first and the second electrodes are proton conducting. In many embodiments, the first and second electrodes comprise sulfonated pentablock copolymer (such as Kraton Polymer's NEXAR). In many embodiments, the first electrode, the second electrode, and the dielectric elastomer membrane are optically transparent. In many embodiments, the dielectric elastomer membrane underlying the active film comprises an electroactive elastomer, such as, for example, acrylic elastomer. In many embodiments, the active film comprises more than one variant or derivative of the nonacene-like molecule, wherein each variant or derivative possesses distinct optical properties or coloration. In many such embodiments, the distinct variants or derivatives are arranged in a design or pattern over the active film. In many embodiments, the at least one variant or derivative of the nonacene-like molecule is TBN. In many embodiments, the at least one variant or derivative of the nonacene-like molecule is PTBN. However, in some embodiments, the active film comprises a patterned combination of TBN and PTBN. In many such embodiments, the patterned combination comprises a blue annulus comprising TBN surrounding a brown circle comprising PTBN.

Accordingly, in many embodiments, prior to actuation, the DEA device comprises a contracted nano-to-micro-structured active region comprising the nonacene-like molecule characterized by a bright visible appearance, a strong associated near-infrared signature, and a large corresponding fluorescence signal (FIG. 2, left). However, in many embodiments, after actuation, the DEA device comprises expanded active region with flattened nano-to-micro-structures characterized by a lighter visible appearance, a weaker associated near-infrared signature, and a smaller corresponding fluorescence signal (FIG. 2, right), as compared to the DEA device prior to actuation. As such, in many embodiments, the instant DEA device possesses color- and appearance-changing capabilities similar to those of the H. lunulata octopus' blue rings, in addition to a number of other highly desirable capabilities and metrics as described below herein.

### Nonacene-Like Molecule

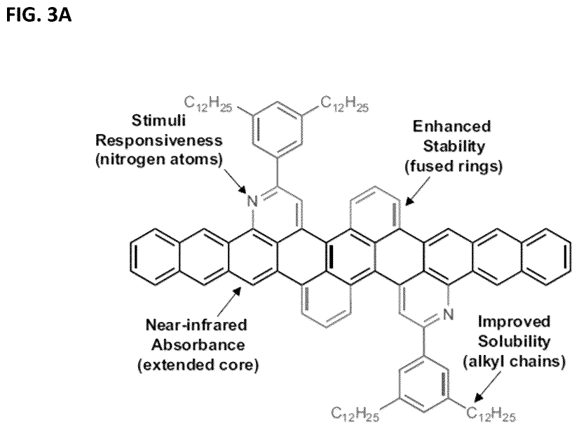

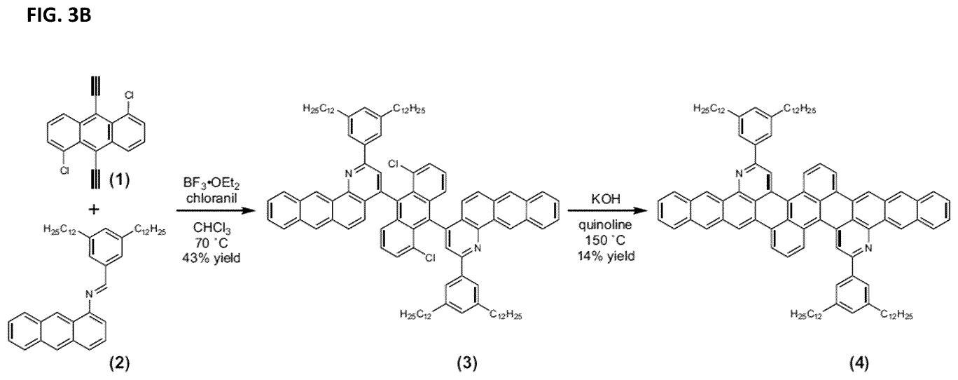

In many embodiments, the active material for the DEA device is a nonacene-like molecule (an acene) characterized by a unique basic architecture shown, for example, in FIG. 3A. To this end, the nonacene-like molecule comprises:
1) a molecular core, wherein the molecular core is an extended, pi-conjugated, all-carbon nonacene core (i.e., nine ortho-annulated benzene rings in a linear arrangement, which furnish a relatively low bandgap and, thus, a red-to-near-infrared absorbance;
2) four peripheral fused aromatic rings added to expand the molecular core as shown in FIG. 3A (gray), and, thus, to substantially enhance stability of TBN through introduction of multiple aromatic sextets;
3) one or more nitrogen heteroatoms incorporated within one, two, three, or all four of the four peripheral aromatic rings to afford responsiveness to chemical stimuli (e.g., protonation); and
4) optionally, one or more pendant, alkyl chain-functionalized phenyl rings on one, two, three, or all four peripheral aromatic rings, such as to mitigate intramolecular pi-pi stacking and improve solubility of the nonacene-like molecule in different solvents.

Accordingly, in many embodiments, the nonacene-like molecule is TBN, including its derivatives such a N-protonated TBN (PTBN). However, in some embodiments, the nonacene-like molecule comprises a molecular core that is shorter or longer than nine benzene rings, comprises less or more peripheral aromatic rings, and features distinct from nitrogen and or additional heteroatoms (such as, for example, an atom selected from the group consisting of: N, B, O, S, P, Si, and any combination thereof) on the peripheral aromatic rings, as long pi-conjugation within the molecule and the corresponding visible to NIR optical and spectroscopic properties and stability are maintained. As such, in many embodiments, the molecular architecture of the acene molecule descried herein allows for advantageous spectroscopic properties, ambient-atmosphere stability, and good solubility in various solvents—that is, essential material selection criteria for camouflage or other demanding device applications.

In many embodiments, nonacene-like molecule is readily available via a facile synthetic strategy schematically illustrated in FIG. 3B for the TBN-specific example. In some such embodiments, the synthetic strategy for obtaining nonacene-like molecules relies on modified known methodologies for the preparation and characterization of nitrogen-containing tetrabenzopentacenes, polydiquinolineanthracenes, and zigzag polyquinolines. More specifically, in some embodiments, first, 1,5-dichloro-9,10-diethynylanthracene (1) and didodecyl-substituted phenylmethanimine anthracene (2) precursors (FIG. 3B, left) are obtained according to known procedures (as also provided in greater detail in the Exemplary Embodiments section below). Next, in many embodiments, the key dinaphthoquinolineanthracene (3) intermediate is generated by reacting precursors (1) and (2) via the Lewis acid-mediated aza-Diels-Alder (Povarov) reaction (FIG. 3B, middle; and as also described in more detail in the Exemplary Embodiments section below). Finally, in many embodiments, intermediate (3) is converted into the desired nonacene-like molecule TBN (4) in moderate yield by forming internal C—C bonds between the anthracene core and its two pendant naphthoquinolines via a base-mediated cyclodehydrohalogenation reaction (FIG. 3B, right; and as also described in greater detail in the Exemplary Embodiments section below). Accordingly, in many embodiments, TBN (4) is synthesized by employing straightforward reaction conditions, and isolated in reasonable yields.

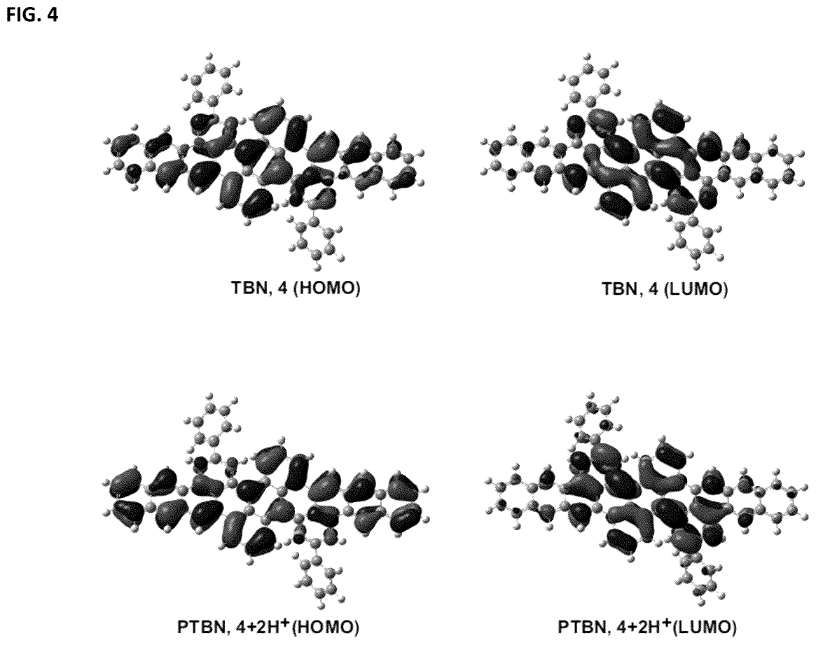

In addition, FIG. 4 provides computational insight into the likely conformation and electronic properties of the nonacene-like molecule, according to many embodiments. More specifically, in many embodiments, the molecular geometries predicted for TBN and PTBN by using standard DFT reveal twisted concave nonacene cores and downward oriented tilted phenyl rings, suggesting that solubility would be enhanced due to disruption of intermolecular pi-pi stacking interactions. (Here, the ground-state geometries, electronic structures, and molecular orbitals for both the unprotonated tetrabenzononacene (TBN, 4) and the protonated tetrabenzononacene (PTBN, 4+2H⁺), excluding the side chains for computational tractability, were calculated by employing conventional density functional theory (DFT) and unrestricted broken symmetry DFT with the B3LYP functional and the 6-311G (d,p) basis set) as discussed, among others. Furthermore, in many embodiments, the corresponding highest occupied molecular orbitals (HOMOs) and lowest unoccupied molecular orbitals (LUMOs) calculated for TBN and PTBN using standard DFT and provided in FIG. 4, feature comparable shapes and nearly encompass the pi-conjugated cores, albeit with reduced LUMO electron densities at the molecular termini. In many embodiments, the HOMO and LUMO energies are −5.01 eV and −3.11 eV, respectively, for TBN, whereas the HOMO and LUMO energies are −9.59 eV and −8.07 eV, respectively, for PTBN, suggesting enhanced oxidation resistance for TBN and PTBN upon protonation. Moreover, in many embodiments, the electronic configurations for TBN and PTBN (as predicted with unrestricted broken symmetry DFT) are closed-shell, suggesting reduced reactivity for TBN and PTBN with respect to the open-shell all-carbon nonacene. Accordingly, in many embodiments, the nonacene-like molecule as exemplified by TBN and its protonated version PTBN possess increased solubility in standard solvents and enhanced stability in ambient atmosphere, as compared to other reported acenes.

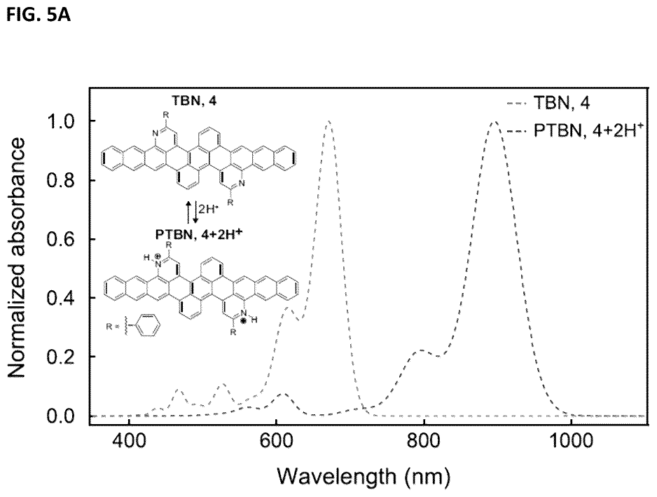

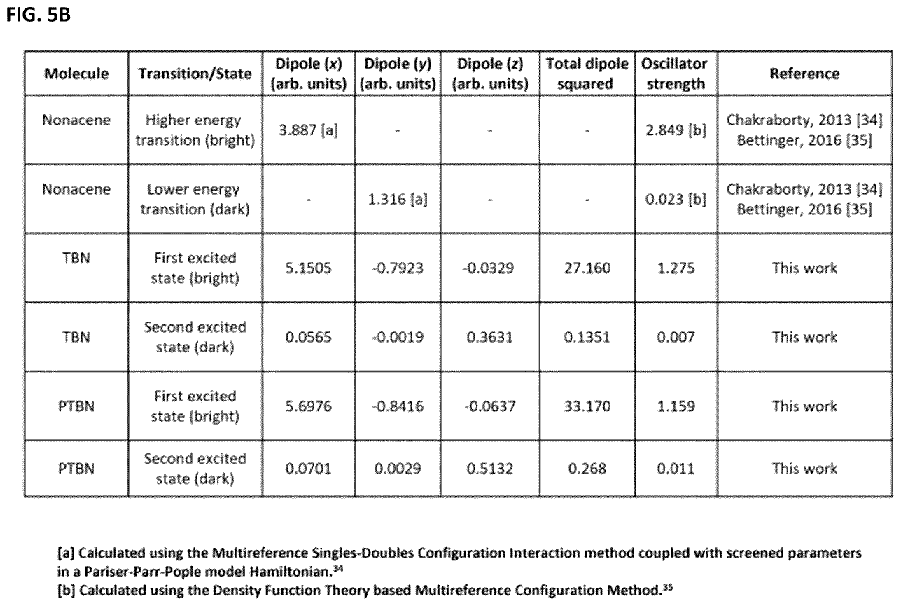

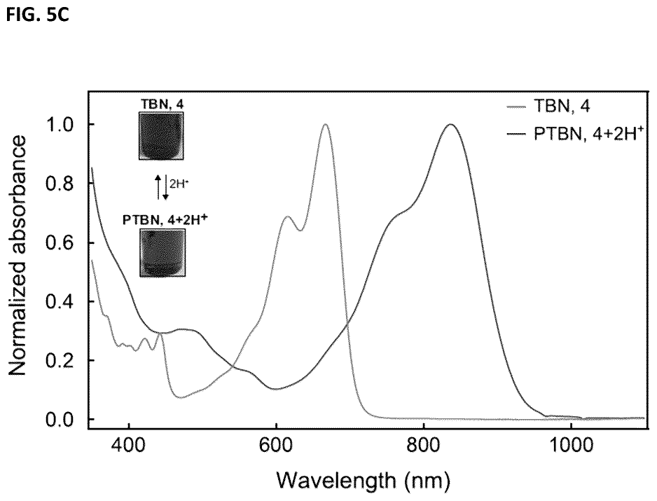

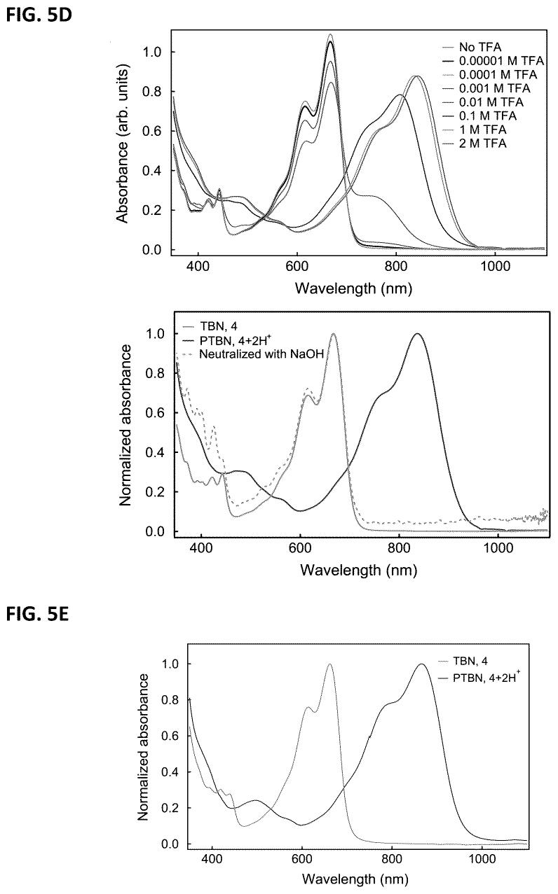

Moreover, FIG. 5A provides computational insight into the optical properties of TBN and PTBN according to many embodiments. In particular, FIG. 5A shows the outcome of simulating the ultraviolet-visible-near-infrared (UV-Vis-NIR) absorption spectra for TBN and PTBN by employing time-dependent density functional theory (TDDFT), together with the Franck-Condon approximation, to evaluate the nature of the associated electronic transitions. As seen from FIG. 5A, and according to mane embodiments, the simulated absorption spectrum for TBN indicates an onset at ~725 nm, a main peak at ~670 nm resulting from the first two excited states, a large shoulder at ~632 nm resulting from the vibronic progression of the two lower-energy excited states, and multiple smaller peaks between ~550 nm and ~420 nm corresponding to the three higher-energy excited states. Furthermore, as seen from FIG. 5A, and according to mane embodiments, the simulated absorption spectrum for PTBN indicates an onset at ~1000 nm, a main peak at ~896 nm resulting from the two lower-energy excited states, a large shoulder at ~798 nm resulting from the vibronic progression of the first two excited states, and multiple smaller peaks between ~650 nm and ~520 nm corresponding to the three high energy excited states. Notably, as seen from the simulated data, the absorption spectrum predicted for PTBN is red-shifted by >200 nm relative to the one predicted for TBN, in agreement with theoretical and experimental precedent for N-heteroacenes (although not to be bound by any theory). Also notably, the bright first excited states and dark second excited states calculated for TBN and PTBN are polarized along and orthogonal to the nonacene core, in contrast to computational findings for the parent all-carbon molecule (as seen from the Table provided in FIG. 5B). Accordingly, in many embodiments, TBN absorbs light in the red-to-near-infrared regions of the electromagnetic spectrum and a routine chemical stimulus (such as, e.g., protonation to produce PTBN) shifts its absorbance further into the near infrared range.

In addition, FIG. 5C illustrates optical properties of the nonacene-like molecule as exemplified by TBN and PTBN in solution-phase according to many embodiments. More specifically, to collect this data, solutions of TBN of many embodiments were first prepared by dissolving the compound in various organic solvents, and, next, TBN solutions were converted into PTBN solutions through the addition of acid, before interrogating both types of solutions with UV-Vis-NIR spectroscopy in ambient atmosphere. As seen from FIG. 5C, and according to many embodiments, the absorption spectra for TBN solubilized in chloroform demonstrates an onset at ~708 nm, a major peak with a maximum at ~667 nm, and a cluster of sharp small peaks between ~600 nm and ~360 nm. In addition, in many embodiments, solutions of TBN in chloroform and other solvents feature a bright blue color at high molecular concentrations (FIG. 5C, inset). As further seen from FIG. 5C, and according to many embodiments, the absorption spectra for PTBN solubilized in chloroform demonstrates an onset of ~924 nm, a major peak with a maximum at ~837 nm, and a cluster of broadened small peaks between ~600 nm and ~400 nm. In addition, in many embodiments, solutions of PTBN in chloroform and other solvents feature a dark brown color at high molecular concentrations (FIG. 5C, inset). It should be noted here, that, in many embodiments, the absorption spectra obtained for PTBN are red-shifted by >170 nm relative to those obtained for TBN, as expected from the theoretical predictions (vide supra) and extensive literature precedent for N-heteroacenes. Moreover, in many embodiments, the conversion of TBN into PTBN is dependent on the amount of added acid, reversible with concentrated base, and achievable in multiple solvent/acid systems (as seen, for example in FIGS. 5D and 5E). Notably, for TBN and PTBN of many embodiments, the experimentally obtained spectra are in reasonable agreement with the theoretically predicted spectra, with the differences likely, although not to be bound by any theory, resulting from the limited accuracy of the employed functionals and the influence of challenging-to simulate solvation effects (compare, for example, FIGS. 5A thought 5E). Accordingly, in many embodiments, the nonacene-like molecule, including TBN and PTBN, possesses good solubility, and, in many embodiments, protonation can modulate visible to near-infrared solution absorbance of TBN.

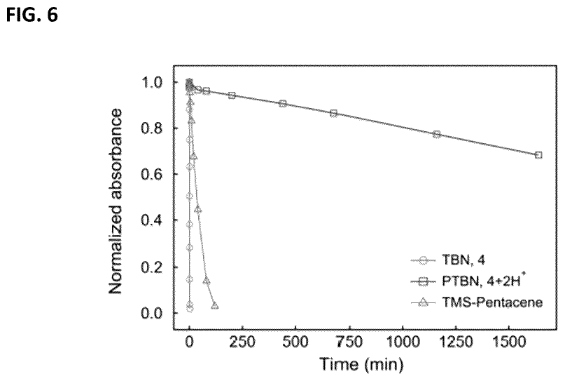

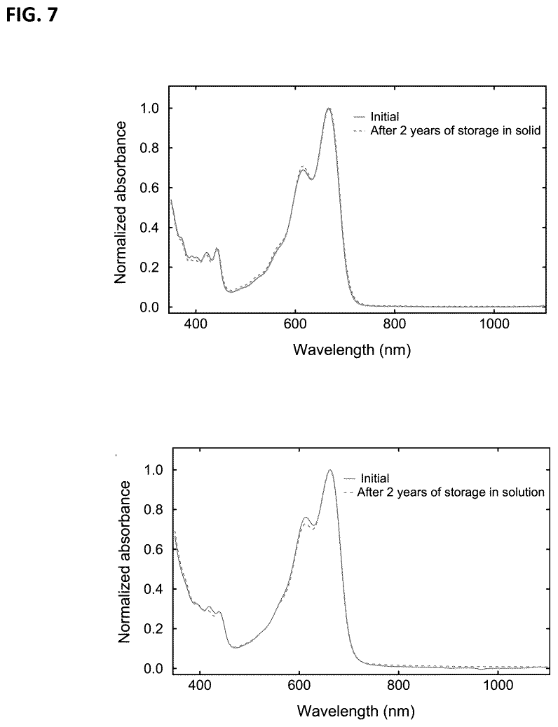

In many embodiments, nonacene-like molecule is stable under many environmental conditions, including conditions that would prove challenging for any acene molecule variant (e.g., including high intensity broadband illumination in ambient atmosphere). Towards this end, FIG. 6 provides data collected in experiments wherein prepared solutions containing TBN, PTBN, or 6,13-bis((trimethylsilyl) ethynyl) pentacene (i.e., TMS-pentacene, which is a classic reference molecule with well-known improved stability), were comparatively characterized with UV-Vis-NIR spectroscopy during high-intensity broadband illumination in ambient atmosphere. In particular, FIG. 6 shows that, under such intentionally harsh conditions, according to many embodiments, TBN's peak absorbance decays relatively rapidly with a half-life of 0.3±0.1 minutes, but PTBN's peak absorbance decays >7800-fold more slowly with a half-life of 2358±307 min. By comparison, TMS-pentacene's peak absorbance decays rapidly with a half-life of 35.2±1.5 min (FIG. 6). Therefore, in many embodiments, both TBN and PTBN are able to withstand environmental conditions that would instantaneously decompose any of the reported to date nonacenes, with PTBN even exhibiting a >65-fold solution-phase stability enhancement relative to the well-known TMS-pentacene reference molecule. Remarkably, and in many embodiments, TBN shows no obvious signs of degradation even after >2 years of storage as a solid or in solution and, thus, demonstrates a >10-fold stability enhancement relative to the reported nonacene derivatives, as confirmed by UV-Vis-NIR spectroscopy, proton NMR spectroscopy, and MALDI mass spectrometry (as shown, for one example, in FIG. 7). Accordingly, in many embodiments, the nonacene-like molecule, exemplified here by TBN and PTBN, possesses outstanding general robustness under extreme for such family of molecules conditions and, therefore, these molecules are particularly good candidates for use as the material base in such demanding applications/technologies as deception and signaling devices and systems.

### Films Comprising the Nonacene-Like Molecule

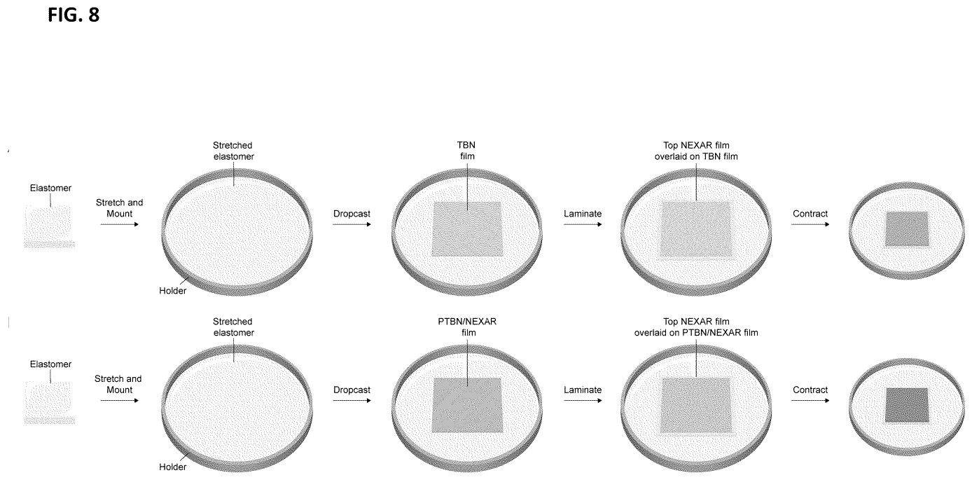

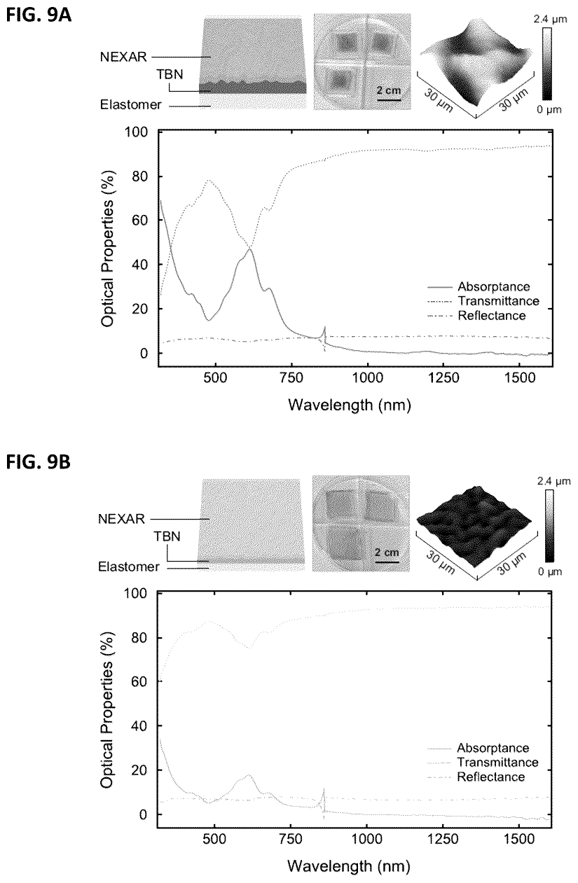

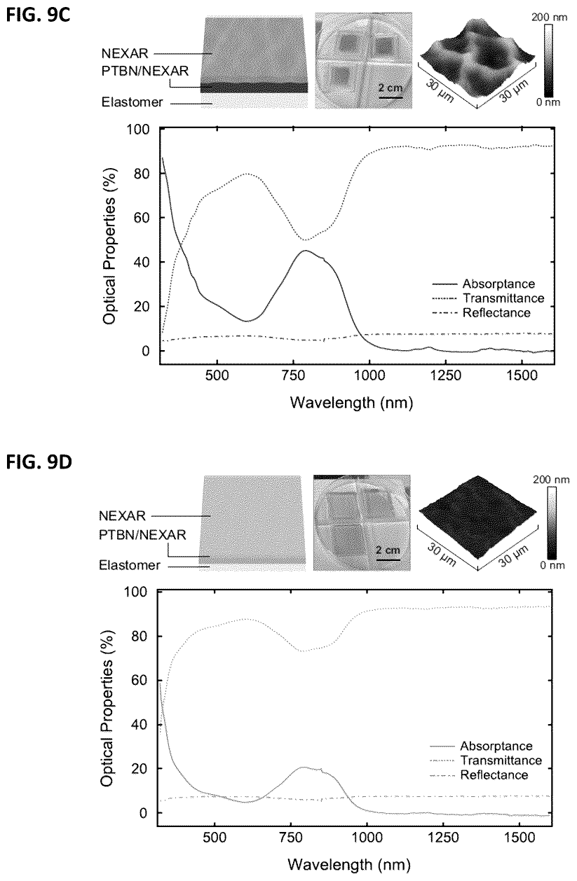

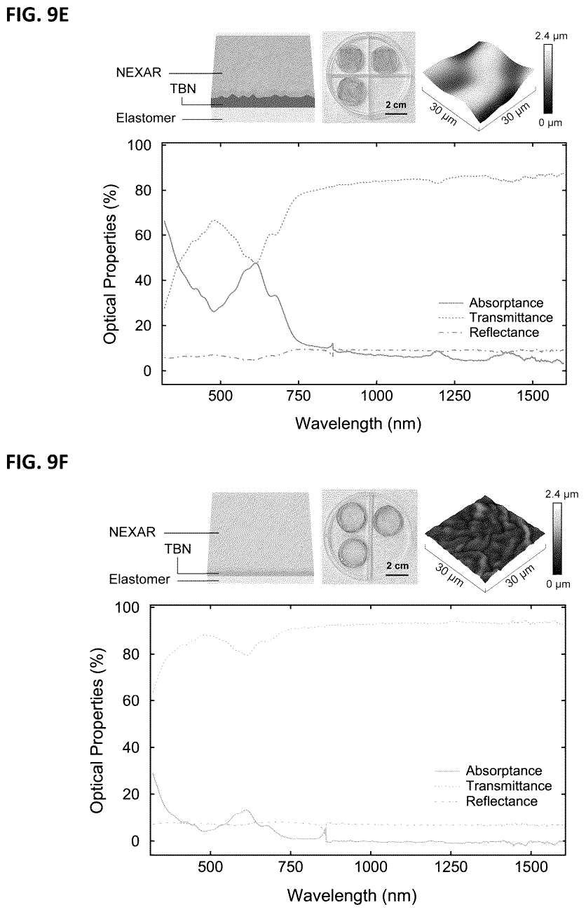

In addition, in many embodiments, films comprising highly soluble, exceptionally stable, and readily processable nonacene-like molecule, such as, for example, TBN and PTBN also possess advantageous solid-state spectroscopic properties, including in device-relevant configurations. For example, FIG. 8 illustrates preparation of such films according to many embodiments, wherein a tri-layer architecture featuring texturizing nano-to-micro-structures (i.e., wrinkles) is fabricated by sandwiching a drop cast neat TBN or mixed PTBN/NEXAR sulfonated pentablock copolymer (NEXAR) (top or bottom, respectively) film between a stretched elastomer layer and a layer of NEXAR film, and allowing so prepared film to contract/relax. Furthermore, FIGS. 9A though 9D, provide images and data illustrating spectroscopic properties of such films of many embodiments characterized with digital camera imaging (DCI), atomic force microscopy (AFM), and UV-Vis-NIR spectroscopy, both with and without mechanical actuation constituting stretching and releasing, respectively, wherein the film's texture switches between more smooth and “wrinkled” and textures, respectively. To this end, FIG. 9A (top) shows that, according to many embodiments, the unactuated films comprising TBN are characterized by a bright blue coloration and a nano-to-micro-structured surface topography. Furthermore, the corresponding spectra shown in FIG. 9A (bottom) reveal, according to many embodiments, absorptances of ~30.6±2.6%, transmittances of ~63.4±2.5%, and reflectances of ~6.0±0.1% at ~676 nm. However, when actuated (i.e., here, stretched), the films comprising the TBN-based layer prepared according to many embodiments are characterized by a lighter blue coloration and >2-fold flatter topography, as seen in FIG. 9B, (top). The corresponding spectra provided in FIG. 9B (bottom) reveal, according to many embodiments, decreased absorptances of ~11.6±1.7%, increased transmittances of ~81.1±1.1%, and comparable reflectances of ~7.2±0.6% at ~676 nm. Similarly, FIG. 9C (top) shows that, according to many embodiments, the unactuated films comprising PTBN are characterized by a dark brown coloration and a nanostructured surface topography. Moreover, the corresponding spectra shown in FIG. 9C (bottom) reveal, according to many embodiments, absorptances of ~43.7±1.2%, transmittances of ~51.3±1.1%, and reflectances of ~5.0±0.1% at ~792 nm. Yet, when actuated/stretched, the film comprising the PTBN-based layer prepared according to many embodiments are characterized by a lighter brown coloration and >3-fold flatter surface topography, as seen in FIG. 9D, (top). The corresponding spectra provided in FIG. 9D (bottom) reveal, according to many embodiments, decreased absorptances of ~21.4±2.4%, increased transmittances of ~72.5±2.3%, and comparable reflectances of ~6.1±0.2% at ~792 nm. As such, according to many embodiments (although not to be bound by any theory), upon actuation/stretching, the strain-induced reduction in both the effective thickness and substantial surface roughness of the TBN or PTBN layer-containing films lowers their absorptance and enhances their transmittance. Notably, in many embodiments, the TBN-containing films demonstrate excellent stability, wherein it has been observed that such films fabricated from TBN solutions that were stored for >2 years feature actuation-dependent visible appearance, surface topology, and spectroscopic characteristics that are similar to those of analogous films fabricated from freshly prepared TBN solutions (compare FIGS. 9A, 9B and 9E, 9F). Accordingly, in many embodiments, nonacene-like molecule is an exceptionally suitable active material for deception and signaling platforms.

### Systems and Devices Comprising the Nonacene-Like Molecule

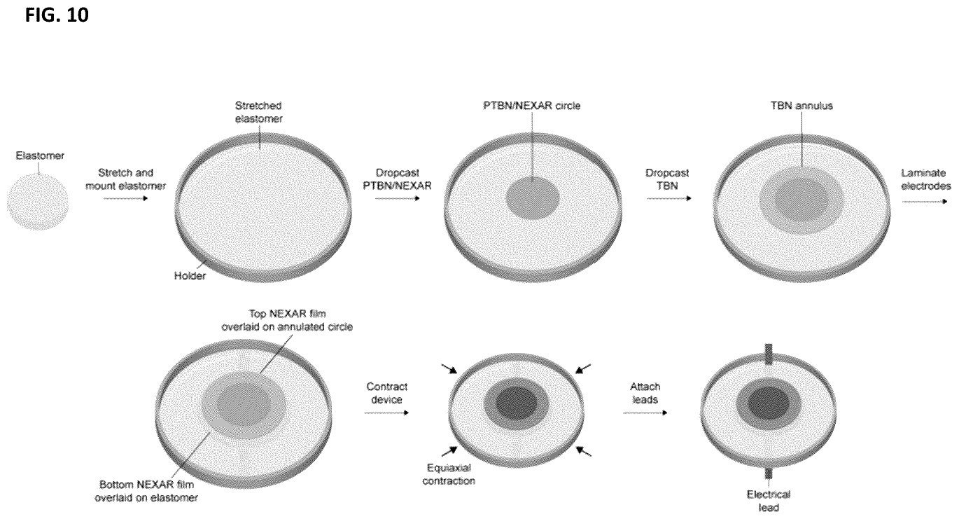

In many embodiments, the nonacene-like molecule is used to manufacture adaptive systems and devices with appearance-changing capabilities within the visible region of the electromagnetic spectrum (inspired by octopus blue ring). As one example, in many embodiments, a DEA device, wherein the DEA device is a quad-layer dielectric elastomer actuator device featuring a plurality of surface-texturizing nano-to-micro-structures/wrinkles, is fabricated such that its active layer, comprising TBN-based blue annulus enclosing a PTBN/NEXAR-based brown circle (i.e., an active area), is sandwiched between a stretched elastomer layer (in turn overlaying a first NEXAR layer electrode) and a second NEXAR layer electrode, and the DEA device is allowed to contract/relax post layering, as schematically illustrated in FIG. 10. More specifically, in many embodiments, the quad-layer DEA device comprises 4 layers that mimic the shape and architecture of the blue-ringed octopus, including, top to bottom, respectively: a first layer comprising a transparent proton-conducting membrane (electrode); a second layer comprising a patterned and or variable-colored film comprising nonacene-like molecule and or its derivatives; a third layer comprising a pliable elastomer (e.g., acrylate), and a fourth layer comprising the transparent proton-conducting membrane (electrode). In many embodiments, the device components/layer are stretched, uniaxiallly or equiaxially, during the fabrication process, such that, when the completed device is released to its relaxed (i.e., unactuated state), it obtains its nano-to-micro-structured texture/wrinkles. In many embodiments, upon actuation (i.e., signal input) the device is stretched, causing flattening of the nano-to-micro-structures and, thus, changing the overall shape and dimensions of the DEA device to enable unique visible and near-IR optical and fluorescent capabilities.

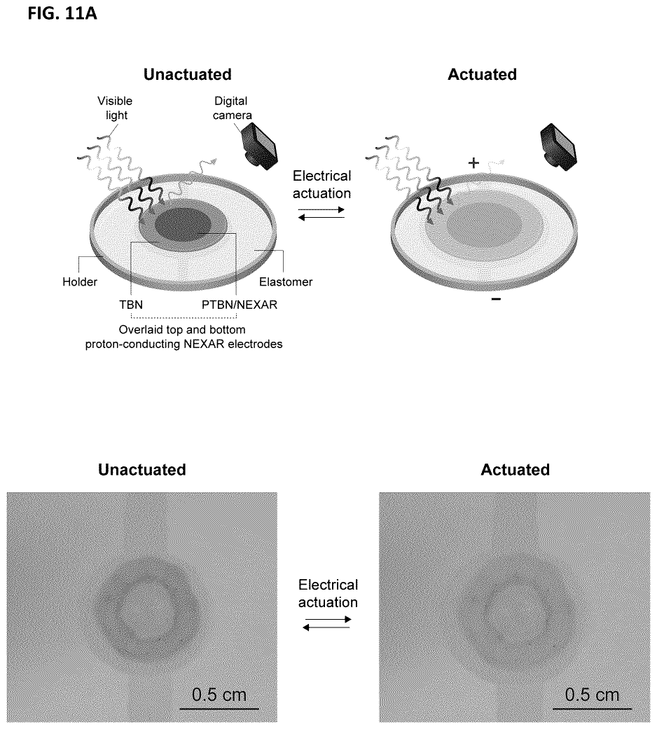

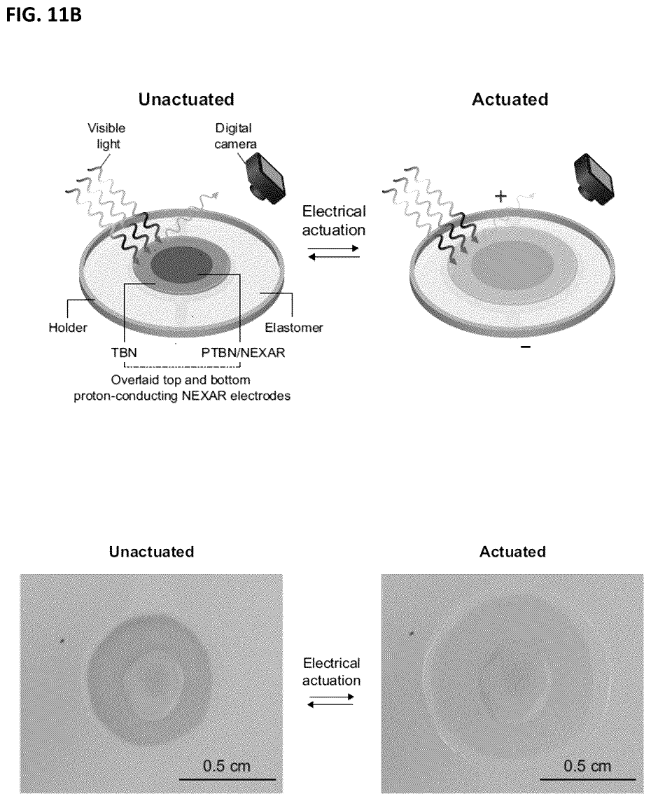

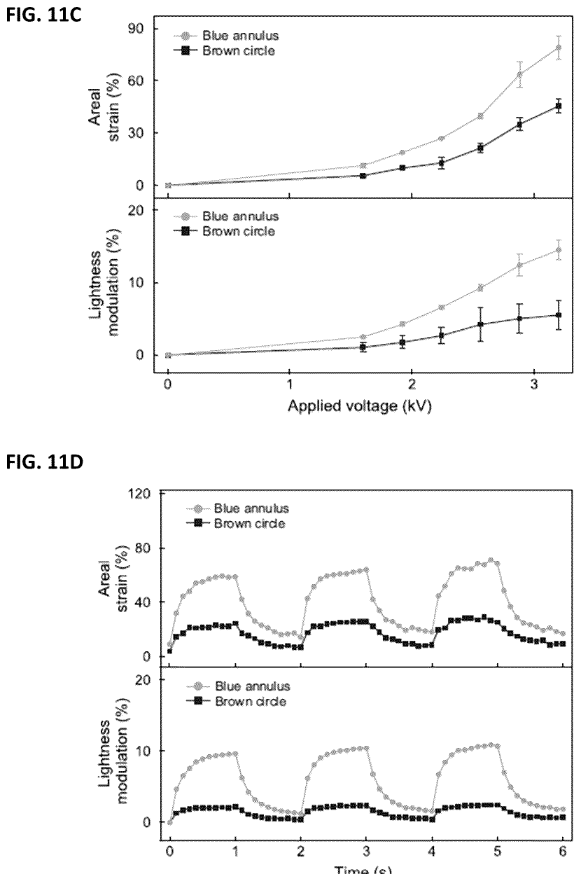

To this end, FIGS. 11A through 11D provide visualization and quantification of the color lightness and areal strain for such DEA device of many embodiments via DCI during variable-bias electrical actuation above a white scattering background under standard indoor lighting in ambient atmosphere. More specifically, as seen from FIGS. 11A and 11B, and according to many embodiments, the blue-brown annulated circle of the DEA device's active area simultaneously expands laterally and becomes noticeably lighter upon actuation (i.e., stretching) with large biases of either ~2.9 kV or ~3.2 kV. Furthermore, in many such embodiments, the areal strain and lightness changes for the active area feature characteristic dependences on the magnitude of the applied voltage as shown in FIG. 11C, with average strains of ~79±6% and ~45±4% and average lightness changes of ~14±1% and ~5±2% for the blue outer annuli and brown inner circles, respectively, at a ~3.2 kV bias. Moreover, in many embodiments, the DEA device exhibits rapid response times of ~380 msec when repeatedly actuated by a 0.5 Hz frequency square waveform with a 0 kV minimum and a ~2.9 kV maximum (FIG. 11D). Accordingly, in many embodiments, the DEA device of many embodiments comprising the nonacene-like molecule as the active material possesses dynamic visible appearance-changing capabilities and is characterized by operational performance metrics that compare favorably to those reported for any similar camouflage platform reported to date.

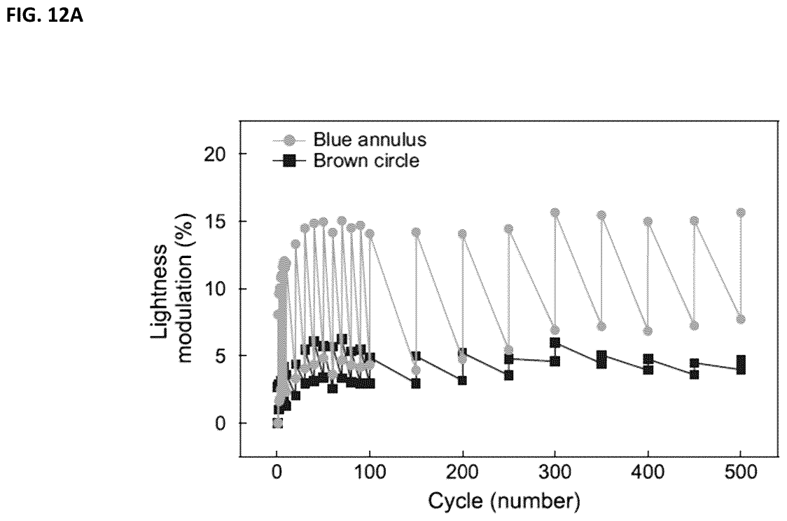

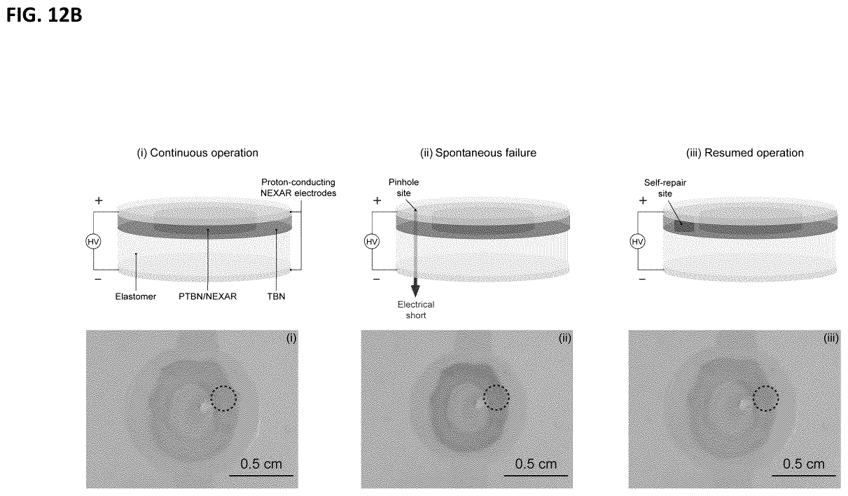

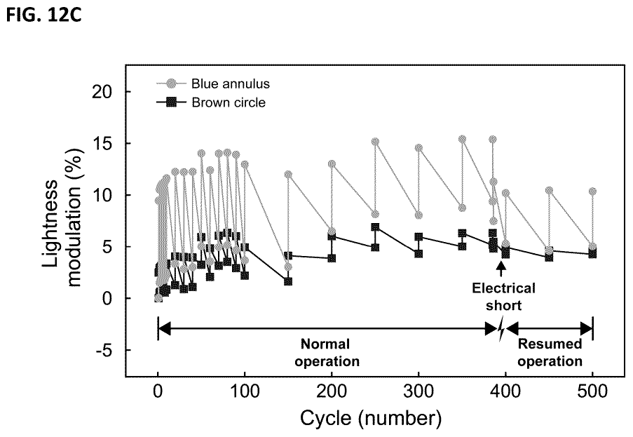

In many embodiments, the adaptive systems and devices with appearance-changing capabilities are exceptionally stable under relevant operation conditions. For example, FIGS. 12A through 12C provide data illustrating systematical characterization of the color lightness of 10 different quad-layer DEA devices of many embodiments via DCI during hundreds of rounds of repeated electrical actuation with a ~2.9 kV bias above a white scattering background under standard indoor lighting in ambient atmosphere. More specifically, in this example, but also according to some embodiments, as seen from the data provided in FIG. 12A, for 7 of the 10 thus tested devices, the blue-brown annulated circle (i.e., the active area) featured uniform coloration and revealed no obvious evidence of physical delamination or electrochemical degradation (FIG. 11A) after continuously operating with stable lightness modulation over at least 500 cycles. However, in the same set of experiments, for 3 of the 10 devices, the active area comprising the blue-brown annulated circle initially featured uniform coloration but then acquired localized discoloration suggestive of chemical degradation (FIG. 12B, middle). Nevertheless, according to the data provided in FIG. 12C, the same 3 devices continuously operated with stable lightness modulation for ~100 to ~400 cycles, spontaneously failed with a corresponding brief pause in functionality, and then resumed operating but with altered lightness modulation for ~50 to ~100 more cycles. Here, although not to be bound by any theory, the DEA devices presumably failed through classical dielectric breakdown and pinhole shorting mechanisms common for dielectric elastomer actuators (and other capacitors) and then likely, and according to some embodiments, self-repaired through local conversion of the nonacene-like molecule and/or the acrylic elastomer layer into an insulating material (FIG. 12B). Accordingly, in many embodiments, the adaptive systems and devices with appearance-changing capabilities described herein are characterized by exceptional ambient atmosphere cycling stability and possess an ability to autonomously self-repair without any user intervention (regardless of the exact mechanism). It should be noted here, that autonomously self-repair is a rare capability among convention al dielectric elastomer actuators and is unprecedented among similar camouflage platform known to date.

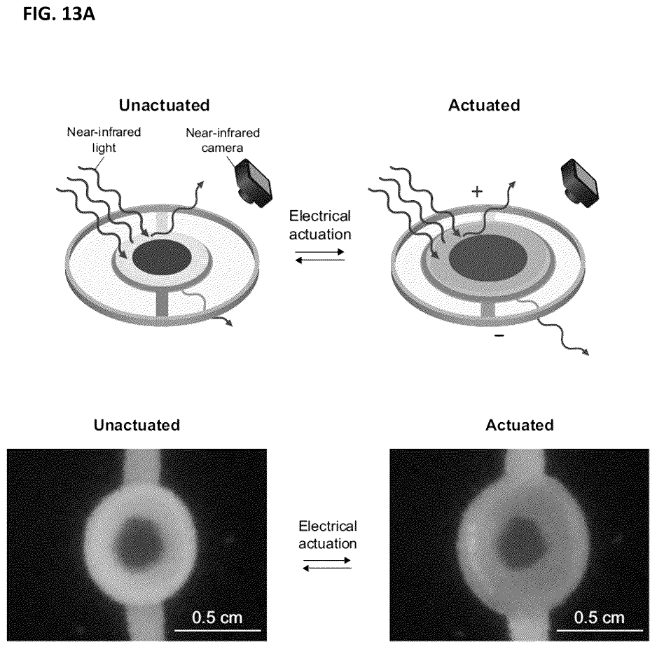

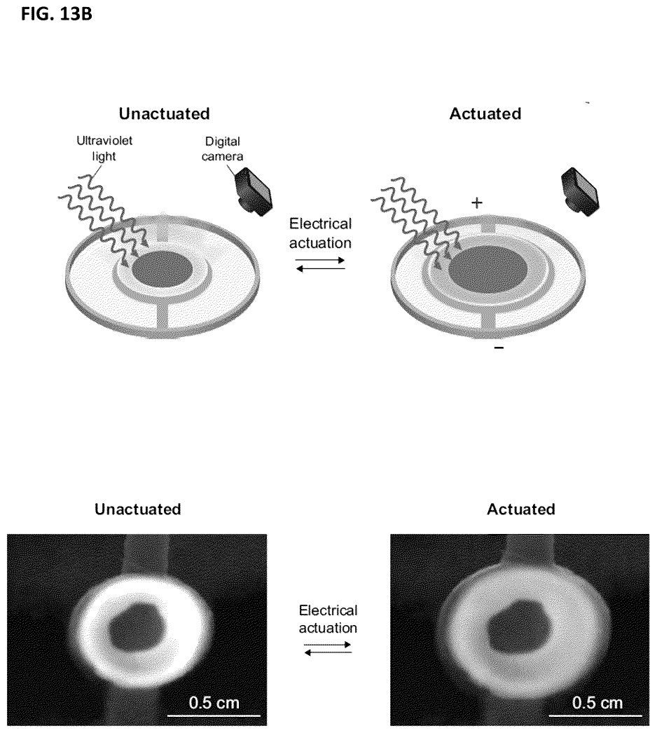

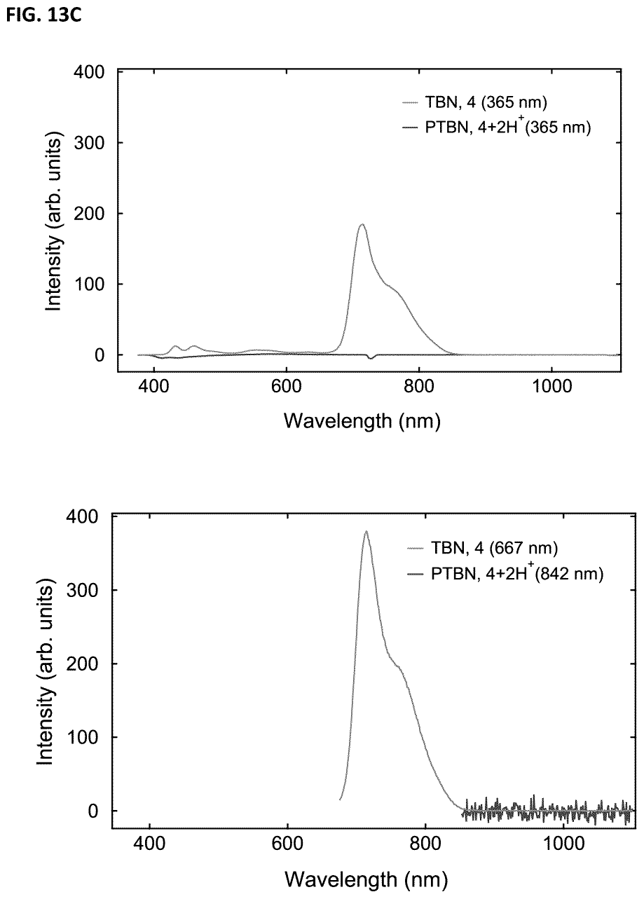

In many embodiments, the adaptive systems and devices with appearance-changing capabilities are capable of signature management, similarly to the octopus blue ring in nature, within the near infrared region of the electromagnetic spectrum. As one example, FIG. 13A illustrates such capabilities for the quad-layer DEA device of many embodiments described herein, wherein the device comprises the blue-brown/TBN-PTBN annulated circle/active area, by providing data for experiments wherein the near-infrared contrast and areal strain for the device were visualized and quantified via spectrally filtered DCI during electrical actuation above a black absorbing background under near-infrared illumination in ambient atmosphere. To this end, FIG. 13A shows that, according to many embodiments, the annulated circle/active area of the device expands laterally and decreases in contrast with respect to the surroundings upon actuation with a large bias of ~3.2 kV. Furthermore, in this example, and in many embodiments, the active region's outer annulus and inner circle feature average areal strains of ~73±3% and ~46±2% and average contrast changes of ~−27±3% and ~−19±5%, respectively, at a ~3.2 kV bias (FIG. 13A). In addition, and according to many embodiments, the device demonstrates robust and consistent near-infrared contrast changes during multiple sequential actuation cycles. In these experiments, as in many embodiments, the contrast switching observed for the TBN-based outer annulus and PTBN-based inner circle presumably (although not to be bound by any theory) resulted primarily from modulation of its surface roughness-induced scattering and near infrared absorbances (as illustrated by FIGS. 9A through 9F). Accordingly, in many embodiments, the adaptive systems and devices comprising the active areas further comprising the nonacene-like molecule possess near-infrared deception capabilities.

Moreover, in many embodiments, the adaptive systems and devices with appearance-changing capabilities are capable of signature management, similarly to the octopus blue ring, within the visible to near-infrared regions of the electromagnetic spectrum. As an example, FIG. 13B illustrates such capabilities for the quad-layer DEA device of many embodiments described herein, wherein the device comprises the blue-brown/TBN-PTBN annulated circle/active area, by providing data for experiments wherein the fluorescence signal intensity and areal strain for the device were visualized and quantified via DCI during electrical actuation above a white scattering background under ultraviolet illumination in ambient atmosphere. To this end, FIG. 13B shows that, according to many embodiments, the annulated circle expands laterally and decreases in signal intensity with respect to the surroundings upon actuation with a large bias of ~3.2 kV. Furthermore, in this example, as in many embodiments, the active region's outer annulus and inner circle feature average areal strains of ~74±4% and ~43±4% and average signal intensity changes of ~−18±1% and ~−6±2%, respectively, at a ~3.2 kV bias (FIG. 13B). In addition, and according to many embodiments, the device demonstrates robust and consistent fluorescence signal intensity changes during multiple sequential actuation cycles. In these experiments, as in many embodiments, the TBN-based outer annulus exhibited larger signal intensity switching with respect to the PTBN-based inner circle because (although not to be bound by any theory) TBN featured a much stronger visible to near-infrared fluorescence relative to PTBN under ultraviolet (or other) excitation (as seen in FIG. 13C). Accordingly, in many embodiments, the adaptive systems and devices comprising the active areas further comprising the nonacene-like molecule exhibit desirable multispectral signaling capabilities and possess robustness and stability under relevant operational conditions, including under challenging and deleterious continuous ultraviolet irradiation.

Accordingly, in many embodiments, the nonacene-like molecule is a readily synthesized, theoretically tractable, highly soluble, and exceptionally stable yet optically and spectroscopically active material. In particular, in many embodiments, the nonacene-like molecule, wherein the nonacene-like molecule is a functionalized and expanded acene, is obtained via a straightforward, solution-phase synthetic route under accessible reaction conditions affording tens-to-hundreds of milligrams yields. In many embodiments, the nonacene-like molecule features tunable electronic, optical, and physical properties, which are in good agreement with theoretical predictions (vide supra), thus establishing a conceptual framework for the continued engineering and improvement of these highly desirable characteristics. In many embodiments, the nonacene-like molecule is color-tunable over a broad wavelength range. In many embodiments, the nonacene-like molecule demonstrates exceptional stability, including the ability to survive direct high-intensity illumination for over a full day when protonated (representing a >65-fold improvement relative to a well-known acene standard), and the ability to withstand solution-phase or solid-state storage for >2 years with no obvious degradation (representing a >10-fold improvement relative to the reported nonacene derivatives). In many embodiments, the nonacene-like molecule exhibits good solubility at high concentrations in different solvents, presumably (although not to be bound by any theory) due to its twisted aromatic core and pendant alkyl chain-functionalized phenyl rings, and, thus, it can be directly processed into large-area films via standard techniques even after >2 years of storage. As such, in many embodiments, the nonacene-like molecule possesses an outstanding combination of characteristics that objectively improve upon and advance comparable acene-based materials.

Furthermore, in many embodiments, the favorable physical properties of the nonacene-like molecule make it a suitable candidate for applications as an active material in multifunctional, electromechanical, color- and appearance-changing, adaptable systems, platforms and devices, including nature-inspired or other deception and signaling systems, platforms, and devices. In particular, in many embodiments, such adaptable systems comprising the nonacene-like molecule are amenable to fabrication via routine benchtop techniques using minimal equipment and are amenable to large area fabrication. In many embodiments, the adaptable systems comprising the nonacene-like molecule are also exceptionally stable under relevant operation conditions. In addition, in many embodiments, the baseline performance of the adaptable systems compares favorably to that reported for any similar camouflage platform, with figures of merit that include maximum areal strains >~90% and response times of <~400 ms. Furthermore, in many embodiments, the adaptable systems are capable of consistently and reliably changing their visible appearance for ~500 cycles, or more, with minimal-to-no degradation in functionality under ambient conditions. In many embodiments, the adaptable systems possess the ability to autonomously self-repair without user intervention in the event of a sudden electrical breakdown or other catastrophic failure, which is rare among other dielectric elastomer actuators and not known for analogous camouflage platforms. In many embodiments, the adaptable systems demonstrate a unique combination of capabilities in the UV-Vis-NIR region of the electromagnetic spectrum, including the ability to modulate visible color lightness, change near-infrared contrast, and adjust multispectral fluorescence intensity. In many embodiments, the adaptive systems support actuation via multiple various inputs, such as, for example, dual-mode mechanical and electromechanical actuation with rapid response times. In summary, in many embodiments, the adaptive systems comprising the nonacene-like molecule as the active material feature a powerful and unique combination of advantages with respect to other camouflage technologies, including, but not limited to, as just a few examples: a straightforward fabrication under accessible conditions, competitive baseline performance metrics, robustness during cycling/operation with the capacity for autonomous self-repair, and multiple dynamic multispectral operating modes.

Moreover, in many embodiments, the nonacene-like molecule and the adaptive, color- and appearance-changing systems and devices comprising thereof afford a range of promising scientific and technological opportunities for both organic electronic materials and appearance-changing platforms alike. For example, in many embodiments, the facile solution-phase synthetic access to the family of the nonacene-like molecule, together with computationally validated molecular engineering strategy, allows for synthesis and fundamental investigation of many valuable molecule variants, including long, variably functionalized acenes, which, in turn, are expected to have similarly good ambient-atmosphere stability and exciting length-dependent electronic properties. Moreover, in many embodiments, the advantageous photophysical robustness and solution-phase processability of the nonacene-like molecule make it a suitable active material candidate for applications in optoelectronic systems, such as, for example, light-emitting diodes, solar cells, and field effect transistors. In many embodiments, the facile manufacturing access (as described and highlighted herein) allows for the systems, platforms, and devices comprising the nonacene-like molecule or its variant to be made to have a large area/size, such as 100 cm² or more. In addition, in many embodiments, the facile synthetic access to the nonacene-like molecule variants with different functionalizations and modifications allows for the systems, platforms, and devices comprising the nonacene-like molecule to incorporate additional dynamic modalities, as needed to improve the performance of and or add capabilities to such systems, platforms, and or devices. In many embodiments, the systems, platforms, and or devices comprising the nonacene-like molecule are characterized by long-term cyclical stabilities. In many embodiments, the systems, platforms, and or devices comprising the nonacene-like molecule possess autonomous self-repair capabilities. As such, in many embodiments, the systems, platforms, and or devices comprising the nonacene-like molecule are capable of mitigating classical defect-based failure mechanisms for both dielectric elastomer actuators and color- and appearance-changing platforms. In many embodiments, the nonacene-like molecule described herein and the systems, platforms and devices comprising the same, are utilized in areas as varied as, but not limited to: organic and functional materials, bioinspired and biomimetic photonics, biological and chemical sensing, biomedical imaging and bioelectronics, energy generation and conservation, and reconfigurable soft actuation and robotics. In some embodiments, the nonacene-like molecule is employed to enable systems or platforms with sophisticated layouts, including large-area camouflage systems that incorporate multiple distinct pixel types for signature management applications and small-area displays with high pixel densities for signaling applications.

### EXEMPLARY EMBODIMENTS

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., s or sec, second(s); min, minute(s); h or hr, hour(s); and the like.

### Example 1—Synthesis and Chemical Characterization of the Nonacene-Like Molecule

Materials. All chemicals, solvents, and supplies were purchased from Thermo Fisher Scientific, Teledyne Isco, Sigma Aldrich, Acros Organics, Aurum Pharmatech, Combi-Blocks, Univar Solutions, Gallade Chemical, or Kraton Polymers. Typically, toluene, chloroform, and other solvents were dried with 3 Å molecular sieves and stored under argon. Unless otherwise noted, the glassware was oven dried at 150° C.-200° C., and the reactions were performed under dry argon. When necessary, the silica gel columns/cartridges used for purification were flushed with 1/9 triethylamine/hexanes to deactivate the gel and with hexanes to remove excess triethylamine.

### Example 2—Synthetic Protocols

N-(3,5-didodecylbenzylidene) anthracene-1-amine (2). This precursor was typically prepared according to modified versions of established procedures.1-4 First, a mixture of commercially-available 1-anthyrylamine (2.88 g, 14.9 mmol), previously-reported 3,5-didodecylbenzaldehyde (6.00 g, 13.6 mmol),2,3 glacial acetic acid (~0.1 mL), and molecular sieves (10.0 g) in toluene (200 mL) was combined in a sealed pressure vessel and stirred under argon at 150° C. for 5 days. Next, the reaction mixture was cooled to room temperature and filtered through Celite. Subsequently, the solvent was removed in vacuo. Last, the crude material was dissolved in chloroform and isolated by trituration from methanol to yield a brown oil (6.80 g, 81% yield).

4,4′-(1,5-dichloroanthracene-9,10-diyl)bis(2-(3,5-didodecylphenyl) naphtho[2,3-h]quinoline) (3). This intermediate was typically prepared according to modified versions of established procedures. First, a mixture of previously-reported 1,5-dichloro-9,10-diethynylanthracene (1) (1.08 g, 3.66 mmol), 1 chloranil (1.99 g, 8.09 mmol), synthesized precursor (2) (6.80 g, 11.0 mmol), and boron trifluoride diethyl etherate (BF3·OEt2) (1.34 mL, 10.9 mmol) in chloroform (184 mL) was combined in a reaction vessel and stirred under argon at 70° C. for 48 hours. Next, the reaction mixture was cooled to room temperature and sequentially washed with a saturated aqueous sodium bicarbonate solution (200 mL×3) and water (200 mL×3). Subsequently, the organics were poured through a cotton plug, and the solvent was removed in vacuo. In turn, the obtained solids were dissolved in chloroform and precipitated from methanol. Last, the material was further purified by flash chromatography (100/0 to 70/30 hexanes/chloroform) using silica gel and isolated as two atropisomers, which were triturated with hot ethanol to yield pale yellow solids (2.43 g (3, isomer 1+isomer 2), 43% yield).

2,15-bis(3,5-didodecylphenyl)dibenzo[Im:e1f1]dipyrido[2,3,4-hi:2,3,4-a1b1]nonacene (4). This product was typically prepared according to modified versions of established procedures. First, a mixture of the synthesized intermediate (3) (0.500 g, 0.33 mmol), powdered KOH (2.21 g, 39.3 mmol), and anhydrous quinoline (30 mL) was combined in a reaction vessel, thoroughly degassed for 15 minutes, and stirred under argon at 150° C. for 2 hours. Next, the reaction mixture was cooled to room temperature and poured into chloroform (50 mL). In turn, the resulting mixture was sequentially washed with water (50 mL), an aqueous solution of HCl (2.4 M, 50 mL×3), water (50 mL×3), and a saturated solution of aqueous sodium bicarbonate (50 mL). Subsequently, the organics were poured through a cotton plug, and the solvent was removed in vacuo. Then, the obtained solids were rinsed with methanol. Last, the material was purified by flash chromatography using deactivated silica gel (100/0 to 90/10 hexanes/ethyl acetate) and isolated as a dark blue/green solid (0.068 g, 14% yield).

### Example 3—Computational Analysis of the Nonacene-Like Molecule

Density Functional Theory (DFT) Calculations. The restricted or conventional DFT and unrestricted broken symmetry DFT calculations were performed for the unprotonated tetrabenzononacene (TBN, 4) and the protonated tetrabenzononacene (PTBN, 4+2H+) by using the Gaussian 16 (Revision B.01) software package.5 First, the ground-state geometries, electronic structures, and molecular orbitals were calculated for TBN and PTBN with the restricted B3LYP functional and the 6-311G (d,p) basis set, which has been shown to furnish accurate predictions for comparable acenes and N-heteroacenes (note that the side chains were excluded for computational tractability). 1,6-13 The accuracy of the obtained geometries was verified by ensuring the absence of imaginary frequencies in the normal mode calculations. Second, the energies and molecular orbitals were recalculated for TBN and PTBN with the unrestricted broken symmetry B3LYP functional and the same basis set.8,14 The obtained ground-state energies were within 10-6 eV of those calculated with the restricted B3LYP functional, with no mixing of different electron spin states (i.e., spin contamination), and all of the occupied molecular orbitals were doubly occupied, indicating a closed shell character for the unprotonated and protonated forms of the molecule. These combined calculations yielded the ground-state geometries and electronic structures for TBN and PTBN.

Time-Dependent Density Functional Theory (TDDFT) Calculations. The TDDFT calculations were performed for TBN, 4 and PTBN, 4+2H+ by using the Gaussian 16 (Revision B.01) software package.5 Initially, the ground-state geometries and normal vibrational modes were obtained for TBN and PTBN from the restricted or conventional DFT calculations. By assuming that the excited-state and ground-state vibrational modes were identical, the vibronic spectra were calculated for TBN and PTBN by using the excited-state energy gradient with the vertical gradient (VG) Franck-Condon (FC) method, which has been shown to furnish accurate predictions for analogous pi-conjugated systems at reasonable computational cost.15-18 The FC-TDDFT calculations for TBN and PTBN considered the excitations from the lowest-energy vibrational states of the molecules' electronic ground states to the five electronic excited states within the experimentally-accessible energy range, so the predicted absorption spectra were obtained by averaging the vibronic spectra calculated for these five electronic excited states. The combined calculations yielded the excited-state electronic structures and optical properties of TBN and PTBN.

Additional calculations were performed for TBN and PTBN in order to evaluate the nature of their electronic transitions. For this purpose, the electronic transition density matrix (p!″) nm between the ground state and the excited state was calculated for TBN and PTBN according to the following standard equation:

$$( {p}^{ga} ) nm \text{?} ( {ψ}_{α} \vert {c}_{m}^{†} {c}_{α} \vert {ψ}_{g} ) \qquad ( 1 )$$

$$\text{?} \text{indicates text missing or illegible when filed}$$

where Ψ! is the wavefunction of the ground state, Ψ″ is the wavefunction of the excited state, c #$ is the creation operator, c % is the annihilation operator, n and m are the indices of the different atomic orbital basis functions. 19,20 Here, the application of a unitary transformation resulted in the construction of two new orbitals and simplified the qualitative description of the electronic transitions (i.e., for every hole in the occupied space, there was one corresponding particle in the virtual space).19 This approach yielded the natural transition orbitals associated with the lowest energy excited state transitions for TBN and PTBN, which primarily feature highest occupied molecular orbital (HOMO) and lowest unoccupied molecular orbital (LUMO) character with weights of ~0.97. The calculations provided further insight into the excited state electronic structures and optical properties of TBN and PTBN.

### Example 4—Fabrication, Mechanical Actuation, and Characterization of the Tri-Layer Architectures from the Nonacene-Like Molecule

1. Fabrication of the Tri-Layer Architectures. The tri-layer architectures with TBN- or PTBN-based central layers were fabricated on a benchtop according to modified versions of literature procedures, as illustrated in Supplementary FIG. 19.21,22 First, an acrylic elastomer membrane (VHB 4905, 3M) was mounted on a size-adjustable holder and equiaxially stretched by ~1,600% with respect to its initial area. Next, TBN in a 1:4 (v/v) mixture of Hi Sol 15 Aromatic 150 and VM&P Naphtha 66 solvents (with a typical TBN concentration of ~700 μM and without Any additives) was drop-cast onto the pre-stretched membrane to form a square-shaped, bright blue film (size of ~3.5 cm×~3.5 cm), and the solvent was allowed to completely evaporate. Alternatively, PTBN/NEXAR in a 1:4 (v/v) mixture of Hi Sol 15 Aromatic 150 and VM&P Naphtha 66 solvents (with a typical PTBN concentration of ~350 μM and with the acidic NEXAR™ sulfonated pentablock copolymer additive to ensure protonation of the molecule) was drop-cast onto the pre-stretched membrane to form a square-shaped, dark-brown film (length of ~3.5 cm), and the solvent was allowed to completely evaporate. In tandem, a ~5 weight % NEXAR™ sulfonated pentablock copolymer solution was spin-cast onto a silicon substrate, heat-treated on the substrate at a temperature of 60° C., and then delaminated from the substrate as a transparent film. Subsequently, the square NEXAR™ sulfonated pentablock copolymer film (length of ~3.5 cm) was laminated directly on top of either 1) a square-shaped, bright blue TBN film or 2) a square-shaped, dark brown PTBN/NEXAR film. Last, the tri-layer architectures were mechanically contracted by ~40% with respect to the initial lengths of the square-shaped TBN or PTBN/NEXAR layers. The resulting completed architectures were used for the mechanical actuation experiments.

2. Mechanical Actuation of the Tri-Layer Architectures. The tri-layer architectures with TBN- or PTBN-based central layers were mechanically actuated in a size-adjustable holder, as previously reported. The holder enabled the application and release of equiaxial strain. The lengths of the architectures' square-shaped blue or brown regions were increased by ~67% for their actuated states and were then contracted by ~40% for their unactuated (i.e., initial) states. All of the experiments were performed under standard indoor lighting in ambient atmosphere at room temperature. The unactuated or actuated TBN architectures and unactuated or actuated PTBN/NEXAR architectures were used for the subsequent characterization experiments.

3. Digital Camera Characterization of the Tri-Layer Architectures. The visible appearances of the unactuated and actuated tri-layer architectures with TBN- or PTBN-based central layers were characterized via digital camera imaging. The images were obtained with either a built-in iPhone Digital Camera or with a Canon PowerShot SX520 HS Digital Camera. All of the experiments were performed under standard indoor lighting in ambient atmosphere at room temperature. The imaging experiments were repeated for at least three independent unactuated or actuated TBN architectures and at least three independent unactuated or actuated PTBN/NEXAR architectures. The images were processed and analyzed with the Adobe Photoshop software package.

4. Morphological Characterization of the Tri-Layer Architectures. The topographies of unactuated and actuated tri-layer architectures with TBN- or PTBN-based central layers were characterized with atomic force microscopy (AFM). The images were obtained with an Asylum Cypher ES Atomic Force Microscope (AFM) operating in tapping mode. All of the measurements were performed in ambient atmosphere at room temperature. The morphological characterization experiments were repeated for at least three independent unactuated or actuated TBN architectures and at least three independent unactuated or actuated PTBN/NEXAR architectures. The images were processed and analyzed with the Gwyddion software package.

5. Spectroscopic Characterization of the Tri-Layer Architectures. The spectroscopic properties of unactuated and actuated tri-layer architectures with TBN- or PTBN-based central layers were characterized with UV-Vis-NIR spectroscopy. The measurements were performed with a Jasco V-670 UV-Vis-NIR Spectrophotometer outfitted with a Jasco ILN-925 150 mm integrating sphere. This spectrophotometer featured a rectangular-shaped port with a length of ~0.9 cm and a width of ~1.3 cm in transmission mode and a square-shaped port with a length of ~1.6 cm length in reflection mode, and the unactuated and actuated tri-layer architectures' square shaped blue or brown regions were large enough to completely cover the ports in the transmission and reflection modes. The measurements were performed at normal incidence in transmission mode or at an incidence angle of 5° in reflection mode. The measurements were referenced to Jasco Spectralon standards, as appropriate. The absorptance was calculated from the transmittance and reflectance measurements according to the following standard equation:

$$T ( \% ) + R ( \% ) + A ( \% ) = 100 \% \qquad ( 2 )$$

where T is the total transmittance, R is the total reflectance, and A is the total absorptance.21-23 All of the measurements were performed under standard indoor lighting in ambient atmosphere at room temperature. The UV-Vis-NIR spectroscopy experiments were repeated for at least three independent unactuated or actuated TBN architectures and at least three independent unactuated or actuated PTBN/NEXAR architectures. The spectra were processed and analyzed with the Jasco Spectra Manager™, Igor Pro, and Python software packages.

### Example 5—Fabrication, Electrical Actuation, and Characterization of the Quad-Layer Devices from the Nonacene-Like Molecule

1. Fabrication of the Quad-Layer Devices. The TBN- and PTBN-based quad-layer devices were fabricated on a benchtop according to modified versions of literature procedures, as illustrated in Supplementary FIG. 21.21,22 First, an acrylic elastomer membrane (VHB 4905, 3M) was mounted on a size-adjustable holder and equiaxially stretched by ~1,600% with respect to its initial area. Next, PTBN/NEXAR in a 1:4 (v/v) mixture of Hi Sol 15 Aromatic 150 and VM&P Naphtha 66 solvents was dropcast onto the pre-stretched membrane to form a circular, dark-brown active region (typical diameters of ~0.5 cm), with the solvent allowed to completely evaporate. Additionally, TBN in a 1:4 (v/v) mixture of Hi Sol 15 Aromatic 150 and VM&P Naphtha 66 solvents was dropcast onto the pre-stretched membrane to form an annular, dark-blue active region (typical inner diameters of ~0.5 cm and typical outer diameters of ~1.1 cm), with the solvent allowed to completely evaporate. In tandem, NEXAR™ sulfonated pentablock copolymer solutions were spincast onto silicon substrates, heat-treated on the substrates at a temperature of 60° C., and then delaminated from the substrates as transparent proton-conducting electrodes. Subsequently, two identical circular proton-conducting polymer electrodes (typical diameters of ~1.2 cm) featuring rectangular extensions were laminated directly on top of the blue-brown annulated circle region and on the bottom of the elastomer membrane. In turn, the quad-layer structure was mechanically contracted by ~40% with respect to the initial diameters of the two circular electrodes. Last, two identical rectangular aluminum foil electrical leads were connected to both the top and bottom proton-conducting polymer electrodes. The resulting completed devices were used for the electrical actuation experiments.

2. Electrical Actuation of the Devices. The TBN- and PTBN-based quad-layer devices were electrically actuated in a size-adjustable holder, as previously reported. The actuation was performed with a home-built high-voltage power supply consisting of a Stanford Research DS345 function generator, Texas Instruments OPA 548 an operational amplifier, and a EMCO E80 high voltage converter. This voltage source enabled the application of variable-voltage and variable frequency waveforms during monitoring of the devices with either a built-in iPhone Digital Camera or with a Canon PowerShot SX520 HS Digital Camera. The areal strain was calculated from the obtained movies and images according to the following equation:

$$Areal Strain ( \% ) = ( A 1 - A 0 ) / A 0 \times 100 \% \qquad ( 3 )$$

where A0 is the area of the active region before actuation and A1 is the area of the active region after actuation. All of the measurements were performed under standard indoor lighting in ambient atmosphere at room temperature. The electrical actuation experiments were repeated for at least three (and as many as ten) independent devices in all instances. The obtained movies and images were processed and analyzed with the Apple QuickTime Player, MATLAB, and Adobe Photoshop software packages.

3. Visible Characterization of the Devices. The visible appearance of the TBN- and PTBN-based quad-layer devices was characterized both without and with actuation via digital camera imaging, as illustrated in FIG. 11A, 11B. The movies and images were obtained with either a built-in iPhone Digital Camera or with a Canon PowerShot SX520 HS Digital Camera. The visible lightness modulation for our devices' active regions (i.e., blue annuli or brown circles) were calculated from the obtained digital camera images according to the following equation:

$$Lightness modulation ( \% ) = \frac{\begin{matrix}image pixel values ( final state ) - \\ image pixel values ( initial state )\end{matrix}}{image pixel values ( initial state )} \qquad ( 4 )$$

The mean pixel values required for these calculations were auto-calculated from the RGB channels of the images using the histogram function in the Adobe Photoshop software package. For the lightness modulation as a function of the applied voltage in FIG. 4C, the initial state pixel values were extracted from images of the unactuated devices at 0 kV; for the lightness modulation as a function of time in FIG. 4D, the initial state pixel values were extracted from images of the devices at 0 sec; and for the lightness modulation as a function of the cycle number in FIG. 4E, the initial state pixel values were extracted from images of the devices at cycle number zero. The experiments were performed for devices positioned above a white printer paper background under standard indoor lighting in ambient atmosphere at room temperature. The imaging experiments were repeated for at least three (and as many as ten) independent devices. The obtained movies and images were processed and analyzed with the Apple QuickTime Player, MATLAB, and Adobe Photoshop software packages.

4. Near-Infrared Characterization of the Devices. The near-infrared contrast of the TBN and PTBN-based quad-layer devices was characterized both without and with actuation via near infrared camera imaging, as illustrated in FIG. 5A. The movies and images were obtained with a Raspberry Pi NoIR Camera Module v2 outfitted with a Hoya RM-72 infrared filter. The near infrared contrast changes for our devices' active regions (i.e., annuli or circles) were calculated from the obtained near-infrared camera images according to the following equation:

$$Contrast change ( \% ) = \frac{\begin{matrix}image pixel values ( final state ) - \\ image pixel values ( initial state )\end{matrix}}{image pixel values ( initial state )} \qquad ( 5 )$$

The mean pixel values required for these calculations were auto-calculated from the RGB channels of the images using the histogram function in the Adobe Photoshop software package. The initial and final state contrast values were extracted from images of the unactuated and actuated devices, respectively. The experiments were performed for devices positioned above a black benchtop background with illumination from a custom-built near-infrared Xtra LED array in ambient atmosphere at room temperature. The imaging experiments were repeated for at least three independent devices. The obtained movies and images were processed and analyzed with the Apple QuickTime Player, MATLAB, and Adobe Photoshop software packages.

5. Fluorescence Characterization of the Devices. The fluorescence signal intensity of the TBN- and PTBN-based quad-layer devices was characterized both without and with actuation via digital camera imaging, as illustrated in FIG. 5C. The movies and images were obtained with a Canon PowerShot SX520 HS Digital Camera. The fluorescence signal intensity changes for our devices' active regions (i.e., annuli or circles) were calculated from the obtained digital camera images according to the following equation:

$$Signal intensity change ( \% ) = \frac{\begin{matrix}image pixel values ( final state ) - \\ image pixel values ( initial state )\end{matrix}}{image pixel values ( initial state )} \qquad ( 6 )$$

The mean pixel values required for these calculations were auto-calculated from the RGB channels of the images using the histogram function in the Adobe Photoshop software package. The initial and final state intensity values were extracted from images of the unactuated and actuated devices, respectively. The experiments were performed for devices positioned above a white printer paper background with illumination from an Analytik Jena UVP EL Series ultraviolet lamp in ambient atmosphere at room temperature. The imaging experiments were repeated for at least three independent devices. The obtained movies and images were processed and analyzed with the Apple QuickTime Player, MATLAB, and Adobe Photoshop software packages.

### Doctrine of Equivalents

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the terms “approximately” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

What is claimed is:

1. A nonacene-like molecule with a molecular architecture comprising:
a molecular core, wherein the molecular core is an extended, pi-conjugated, all-carbon nonacene core comprising nine ortho-annulated benzene rings in a linear arrangement;
four peripheral aromatic rings fused onto the molecular core such as to provide stabilizing multiple aromatic sextets;
one or more nitrogen heteroatoms incorporated within one, two, three, or all four of the four peripheral aromatic rings; and
optionally, one or more pendant, alkyl chain-functionalized phenyl rings attached to one, two, three, or all four of the four peripheral aromatic rings.

2. In many embodiments, the nonacene-like molecule is a molecule selected from the group consisting of: TBN, PTBN, and any combination thereof.

3. A color- and appearance-changing film characterized by a surface area and a thickness and comprising:
a first layer comprising an optically transparent dielectric elastomer, wherein the first layer has an unrelaxed state wherein the first layer is elastically deformed beyond a relaxed state;
a second layer overlaying the first layer and comprising a film further comprising a nonacene-like molecule; and
a third layer overlaying the second layer and comprising an optically transparent, ion-conducting elastomer; wherein:
the nonacene-like molecule is an acene with a molecular architecture at least comprising:
a molecular core, wherein the molecular core is an extended, pi-conjugated, all-carbon acene core comprising at least five ortho-annulated benzene rings in a linear arrangement;
a plurality of peripheral aromatic rings fused onto the molecular core such as to provide stabilizing multiple aromatic sextets;
one or more heteroatoms incorporated within one or more of the plurality of the peripheral aromatic rings; and
optionally, one or more pendant, alkyl chain-functionalized phenyl rings attached to one or more of the plurality of the peripheral aromatic rings;
wherein, at least when the first layer is in the relaxed state at least the third layer forms a plurality of geometrically reconfigurable nano-to-micro-structures characterized by a structure density;
wherein, when the first layer is in the relaxed state, the surface area is smaller, the thickness is larger, and the structure density is higher than when the first layer is in the unrelaxed state; and
wherein the surface area, the thickness, and the structure density are altered by the elastic deformation of the first layer into and out of the relaxed state.

4. The color- and appearance-changing film of claim 3, wherein the optically transparent, ion-conducting elastomer is sulfonated pentablock copolymer.

5. The color- and appearance-changing film of claim 3, wherein the nonacene-like molecule comprises five ortho-annulated benzene in the molecular core and four peripheral aromatic rings.

6. The color- and appearance-changing film of claim 3, wherein the nonacene-like molecule comprises nine ortho-annulated benzene in the molecular core and four peripheral aromatic rings.

7. The color- and appearance-changing film of claim 3, wherein the one or more heteroatom is an atom selected from the group consisting of: N, B, O, S, P, Si, and any combination thereof.

8. The color- and appearance-changing film of claim 6, wherein the nonacene-like molecule is a molecule selected from the group consisting of: TBN, PTBN, and any combination thereof.

9. The color- and appearance-changing film of claim 3, wherein the second layer comprises more than one variant or derivative of the nonacene-like molecule, each characterized by distinct optical properties and or coloration.

10. The color- and appearance-changing film of claim 9, wherein the distinct variants or derivatives of the nonacene-like molecule are arranged into a design or pattern over the film.

11. The color- and appearance-changing film of claim 9, wherein the second layer comprises TBN and PTBN arranged into a design or pattern over the film.

12. The color- and appearance-changing film of claim 11, wherein the design comprises a blue annulus comprising TBN encompassing a brown ring comprising PTBN.

13. A system capable of electrically induced color- and appearance-changing comprising:
an active area characterized by a surface area and a thickness and comprising:
a first layer comprising an optically transparent, ion-conducting elastomer;
a second layer overlaying the first layer comprising an optically transparent dielectric elastomer, wherein the second layer has an unrelaxed state wherein the second layer is elastically deformed beyond a relaxed state;
a third layer overlaying the second layer and comprising a film further comprising a nonacene-like molecule; and
a fourth layer overlaying the third layer and comprising the same or different optically transparent, ion-conducting elastomer as the first layer; wherein:
the nonacene-like molecule is an acene with a molecular architecture at least comprising:
a molecular core, wherein the molecular core is an extended, pi-conjugated, all-carbon acene core comprising at least five ortho-annulated benzene rings in a linear arrangement;
a plurality of peripheral aromatic rings fused onto the molecular core such as to provide stabilizing multiple aromatic sextets;
one or more heteroatoms incorporated within one or more of the plurality of the peripheral aromatic rings; and
optionally, one or more pendant, alkyl chain-functionalized phenyl rings attached to one or more of the plurality of the peripheral aromatic rings;
wherein, at least when the second layer is in the relaxed state at least the first and fourth layers form a plurality of geometrically reconfigurable nano-to-micro-structures characterized by a structure density;
wherein, when the second layer is in the relaxed state, the surface area is smaller, the thickness is larger, and the structure density is higher than when the second layer is in the unrelaxed state; and
wherein the surface area, the thickness, and the structure density are altered by the elastic deformation of the second layer into and out of the relaxed state.

14. The system of claim 13, wherein the optically transparent, ion-conducting elastomer is sulfonated pentablock copolymer.

15. The system of claim 13, wherein the nonacene-like molecule comprises five ortho-annulated benzene in the molecular core and four peripheral aromatic rings.

16. The system of claim 13, wherein the nonacene-like molecule comprises nine ortho-annulated benzene in the molecular core and four peripheral aromatic rings.

17. The system of claim 13, wherein the one or more heteroatom is an atom selected from the group consisting of: N, B, O, S, P, Si, and any combination thereof.

18. The system of claim 13, wherein the nonacene-like molecule is a molecule selected from the group consisting of: TBN, PTBN, and any combination thereof.

19. The system of claim 13, wherein the third layer comprises more than one variant or derivative of the nonacene-like molecule, each characterized by distinct optical properties and or coloration.

20. The system of claim 19, wherein the distinct variants or derivatives of the nonacene-like molecule are arranged into a design or pattern over the film.

21. The system of claim 19, wherein the third layer comprises TBN and PTBN arranged into a design or pattern over the film.

22. The system of claim 21, wherein the design comprises a blue annulus comprising TBN encompassing a brown ring comprising PTBN.

23. A method for dynamically changing color and appearance of a system comprising:
providing the system comprising:
an active area characterized by a surface area and a thickness and comprising:
a first layer comprising an optically transparent, ion-conducting elastomer;
a second layer overlaying the first layer comprising an optically transparent dielectric elastomer, wherein the second layer has an unrelaxed state wherein the second layer is elastically deformed beyond a relaxed state;
a third layer overlaying the second layer and comprising a film further comprising a nonacene-like molecule; and
a fourth layer overlaying the third layer and comprising the same or different optically transparent, ion-conducting elastomer as the first layer; wherein:
the nonacene-like molecule is an acene with a molecular architecture at least comprising:
a molecular core, wherein the molecular core is an extended, pi-conjugated, all-carbon acene core comprising at least five ortho-annulated benzene rings in a linear arrangement;
a plurality of peripheral aromatic rings fused onto the molecular core such as to provide stabilizing multiple aromatic sextets;
one or more heteroatoms incorporated within one or more of the plurality of the peripheral aromatic rings; and
optionally, one or more pendant, alkyl chain-functionalized phenyl rings attached to one or more of the plurality of the peripheral aromatic rings;
wherein, at least when the second layer is in the relaxed state at least the first and fourth layers form a plura2lity of geometrically reconfigurable nano-to-micro-structures characterized by a structure density;
wherein, when the second layer is in the relaxed state, the surface area is smaller, the thickness is larger, and the structure density is higher than when the second layer is in the unrelaxed state; and
wherein the surface area, the thickness, and the structure density are altered by the elastic deformation of the second layer into and out of the relaxed state;
actuating the system to elastically deform the second layer such as to produce the color and appearance change of the system.

24. The method of claim 23, wherein actuating comprises applying an electrical bias to the first and fourth layers.
